SEQUENCE LISTING (10 sequences)

SEQ ID NO: 1 - DNA, 4654 bp, Homo sapiens
  CDS (648)..(3113)
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaataaaaaaaaaaaaaaa

SEQ ID NO: 2 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 3 - DNA, 3119 bp, Homo sapiens
  CDS (1)..(2925)
atggccgcccagggagagcccggctacctggcggcgcagtcggaccccggctccaacagcgagcgcagcaccgactccccagtgcccggctccgaggacgacttggtcgccggggcgaccctgcacagcccggagtggagcgaggagcgcttccgcgtggacaggaagaaacttgaggccatgttacaagctgctgctgaagggaaaggcagaagtggggaagacttttttcaaaagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtagcagcaccctcttggcacatgcccgctgactaactgtaaagtggacacaggagatgtatgaacagccttcacagcacaccatccttagcactctgggtgtctggtatcaggaccaaagcattttattcgcacctgtactttatggcaaaaaggaagaagagagagaagatgttcttatgatgtcatacagaacac

SEQ ID NO: 4 - protein, 974 aa, Homo sapiens
MAAQGEPGYLAAQSDPGSNSERSTDSPVPGSEDDLVAGATLHSPEWSEERFRVDRKKLEAMLQAAAEGKGRSGEDFFQKIMEETNTQIAWPSKLKIGAKSKKDPHIKVSGKKEDVKEAKEMIMSVLDTKSNRVTLKMDVSHTEHSHVIGKGGNNIKKVMEETGCHIHFPDSNRNNQAEKSNQVSIAGQPAGVESARVRIRELLPLVLMFELPIAGILQPVPDPNSPSIQHISQTYNISVSFKQRSRMYGATVIVRGSQNNTSAVKEGTAMLLEHLAGSLASAIPVSTQLDIAAQHHLFMMGRNGSNIKHIMQRTGAQIHFPDPSNPQKKSTVYLQGTIESVCLARQYLMGCLPLVLMFDMKEEIEVDPQFIAQLMEQLDVFISIKPKPKQPSKSVIVKSVERNALNMYEARKCLLGLESSGVTIATSPSPASCPAGLACPSLDILASAGLGLTGLGLLGPTTLSLNTSTTPNSLLNALNSSVSPLQSPSSGTPSPTLWAPPLANTSSATGFSAIPHLMIPSTAQATLTNILLSGVPTYGHTAPSPPPGLTPVDVHINSMQTEGKKISAALNGHAQSPDIKYGAISTSSLGEKVLSANHGDPSIQTSGSEQTSPKSSPTEGCNDAFVEVGMPRSPSHSGNAGDLKQMMCPSKVSCAKRQTVELLQGTKNSHLHSTDRLLSDPELSATESPLADKKAPGSERAAERAAAAQQNSERAHLAPRSSYVNMQAFDYEQKKLLATKAMLKKPVVTEVRTPTNTWSGLGFSKSMPAETIKELRRANHVSYKPTMTTTYEGSSMSLSRSNSREHLGGGSESDNWRDRNGIGPGSHSEFAASIGSPKRKQNKSTEHYLSSSNYMDCISSLTGSNGCNLNSSFKGSDLPELFSKLGLGKYTDVFQQQEIDLQTFLTLTDQDLKELGITTFGARRKMLLAISELNKNRRKLFESPNARTSFLEGGASGRLPRQYHSDIASVSGRW

SEQ ID NO: 5 - DNA, 3949 bp, Homo sapiens
  CDS (290)..(1882)
gctttgcgtgcgtgtttgcgtacagcggaggtggcggcgcgggcaggtcggagctcggagctgctgcttctggttctcttgtggccgccgtcgctgtccggctgccttgggctgccgaacagacaaggcgtgggccacagcacctcagaagccgacgcagctcgacgcaggggccggcaggagggtgggcgatcgcgtgtcggagggcgccgcgcgggcaggcgggcgggcgccagagggggaaagaggcgggggcggcgggtcagccgctggccgggccggccggggaatgtcgatgcctgacgcgatgccgctgcccggggtcggggaggagctgaagcaggccaaggagatcgaggacgccgagaagtactccttcatggccaccgtcaccaaggcgcccaagaagcaaatccagtttgctgatgacatgcaggagttcaccaaattccccaccaaaactggccgaagatctttgtctcgctcgatctcacagtcctccactgacagctacagttcagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaagacatgtctgctctgatttcactcaggaaacgtgctcagggggagaagcccttggctggtgctaaaatagtgggctgtacacacatcacagcccagacagcggtgttgattgagacactctgtgccctgggggctcagtgccgctggtctgcttgtaacatctactcaactcagaatgaagtagctgcagcactggctgaggctggagttgcagtgttcgcttggaagggcgagtcagaagatgacttctggtggtgtattgaccgctgtgtgaacatggatgggtggcaggccaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccggccatgaacgtcaatgattctgttaccaaacagaagtttgataacttgtactgctgccgagaatccattttggatggcctgaagaggaccacagatgtgatgtttggtgggaaacaagtggtggtgtgtggctatggtgaggtaggcaagggctgctgtgctgctctcaaagctcttggagcaattgtctacattaccgaaatcgaccccatctgtgctctgcaggcctgcatggatgggttcagggtggtaaagctaaatgaagtcatccggcaagtcgatgtcgtaataacttgcacaggaaataagaatgtagtgacacgggagcacttggatcgcatgaaaaacagttgtatcgtatgcaatatgggccactccaacacagaaatcgatgtgaccagcctccgcactccggagctgacgtgggagcgagtacgttctcaggtggaccatgtcatctggccagatggcaaacgagttgtcctcctggcagagggtcgtctactcaatttgagctgctccacagttcccacctttgttctgtccatcacagccacaacacaggctttggcactgatagaactctataatgcacccgaggggcgatacaagcaggatgtgtacttgcttcctaagaaaatggatgaatacgttgccagcttgcatctgccatcatttgatgcccaccttacagagctgacagatgaccaagcaaaatatctgggactcaacaaaaatgggccattcaaacctaattattacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggttcctcactctagtcactaaagaaggattttactctcccagcccagaaaggtgattctttctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctattgagtttaattttgtcctctaggatttatttctgttgtccaaaaaaaaaaaaaaagaaaagaaaaattaaggagaatttttggtgttaatgctgaggaattgcttgagtggttagttgttaccaatttctcttttgaacctttggagctaaggatgctgagtctagagaaatgctagtctcaagccctgttaagtccctctgtttctagcccgtagttcatagcatcagtgaactggagccacaacagcaaattctatcagctgtgtaccatacagcttgtgctgaaggcgaatttcttgagccattactcagtataaagcactgagttctatctttaggatttatctttaagagcaaatttctggtcagctgtgcttctgcaacctaaaatatttaaagggaggtaggtgtgggcaggaggaggaatgataaattgggccagggcaagaaaaatctagcttcatataatttgtctgggactatacaccctatataatgttagttttacagaagtaatatgacttttgattgctacataccacaaagagtttatgaactgagatcataaagggcaactgatgtgtgaagaaagtagtcagtacatcctggctcatgctctgaaagaatatccagagaggctctctcaaagatcagggagatgtattcccatgccatgcaccctgcttcccagcatttctgcatggtcaagtgagctttatgctcatgagctttaagtatataattatccaggattttaaatcctcaacttgttctagcttgtgatccctcaaagttgggtcatacgttagtgctagatactagaaattttcacttttccactgatcagagagacagacattaaaaacaaaaatagaagaaaggaaagctttcaccctgcagcttcttagcagggaacaattgtcttgccaaaacttttttcccttttctctcccattttcttttacccaatcccttcttactccttgccagtgtgaccatgctttcttctctgtagatgttaacagttaaggcctattttcctcgggcacttaaccaaccaatcagaacaccacatctgttaggggaggtaacctggccaacagtgtatccatcacgttagccctgctggagggaagggacccacattcacctgccctctgacctgccccttgatcccatatctattaccgtgtccataggaataataggtaagggctctgtctctgtcaagccatgtaacaaaggacactgttaaaaaaaaaaaaaagtctggcatcagagggagcatgtggagagcaacttgggaagaacaagttcattttgtattgaatgatttttaatgaatgcaatattaatccttgcagatgagcaataatcattaaaatcgattaaaatgataagaccttaaaaaa

SEQ ID NO: 6 - protein, 530 aa, Homo sapiens
MSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY

SEQ ID NO: 7 - DNA, 5830 bp, Homo sapiens
  CDS (648)..(5636)
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtagcagcaccctcttggcacatgcccgctgactaactgtaaagtggacacaggagatgtatgaacagccttcacagcacaccatccttagcactctgggtgtctggtatcaggaccaaagcattttattcgcacctgtactttatggcaaaaaggaagaagagagagaagatgttcttatgatgtcatacagaacac

SEQ ID NO: 8 - protein, 1662 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEIMEETNTQIAWPSKLKIGAKSKKDPHIKVSGKKEDVKEAKEMIMSVLDTKSNRVTLKMDVSHTEHSHVIGKGGNNIKKVMEETGCHIHFPDSNRNNQAEKSNQVSIAGQPAGVESARVRIRELLPLVLMFELPIAGILQPVPDPNSPSIQHISQTYNISVSFKQRSRMYGATVIVRGSQNNTSAVKEGTAMLLEHLAGSLASAIPVSTQLDIAAQHHLFMMGRNGSNIKHIMQRTGAQIHFPDPSNPQKKSTVYLQGTIESVCLARQYLMGCLPLVLMFDMKEEIEVDPQFIAQLMEQLDVFISIKPKPKQPSKSVIVKSVERNALNMYEARKCLLGLESSGVTIATSPSPASCPAGLACPSLDILASAGLGLTGLGLLGPTTLSLNTSTTPNSLLNALNSSVSPLQSPSSGTPSPTLWAPPLANTSSATGFSAIPHLMIPSTAQATLTNILLSGVPTYGHTAPSPPPGLTPVDVHINSMQTEGKKISAALNGHAQSPDIKYGAISTSSLGEKVLSANHGDPSIQTSGSEQTSPKSSPTEGCNDAFVEVGMPRSPSHSGNAGDLKQMMCPSKVSCAKRQTVELLQGTKNSHLHSTDRLLSDPELSATESPLADKKAPGSERAAERAAAAQQNSERAHLAPRSSYVNMQAFDYEQKKLLATKAMLKKPVVTEVRTPTNTWSGLGFSKSMPAETIKELRRANHVSYKPTMTTTYEGSSMSLSRSNSREHLGGGSESDNWRDRNGIGPGSHSEFAASIGSPKRKQNKSTEHYLSSSNYMDCISSLTGSNGCNLNSSFKGSDLPELFSKLGLGKYTDVFQQQEIDLQTFLTLTDQDLKELGITTFGARRKMLLAISELNKNRRKLFESPNARTSFLEGGASGRLPRQYHSDIASVSGRW

SEQ ID NO: 9 - DNA, 6488 bp, Homo sapiens
  CDS (648)..(4421)
ggcggcggctggaggagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagcaaatccagtttgctgatgacatgcaggagttcaccaaattccccaccaaaactggccgaagatctttgtctcgctcgatctcacagtcctccactgacagctacagttcagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaagacatgtctgctctgatttcactcaggaaacgtgctcagggggagaagcccttggctggtgctaaaatagtgggctgtacacacatcacagcccagacagcggtgttgattgagacactctgtgccctgggggctcagtgccgctggtctgcttgtaacatctactcaactcagaatgaagtagctgcagcactggctgaggctggagttgcagtgttcgcttggaagggcgagtcagaagatgacttctggtggtgtattgaccgctgtgtgaacatggatgggtggcaggccaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccggccatgaacgtcaatgattctgttaccaaacagaagtttgataacttgtactgctgccgagaatccattttggatggcctgaagaggaccacagatgtgatgtttggtgggaaacaagtggtggtgtgtggctatggtgaggtaggcaagggctgctgtgctgctctcaaagctcttggagcaattgtctacattaccgaaatcgaccccatctgtgctctgcaggcctgcatggatgggttcagggtggtaaagctaaatgaagtcatccggcaagtcgatgtcgtaataacttgcacaggaaataagaatgtagtgacacgggagcacttggatcgcatgaaaaacagttgtatcgtatgcaatatgggccactccaacacagaaatcgatgtgaccagcctccgcactccggagctgacgtgggagcgagtacgttctcaggtggaccatgtcatctggccagatggcaaacgagttgtcctcctggcagagggtcgtctactcaatttgagctgctccacagttcccacctttgttctgtccatcacagccacaacacaggctttggcactgatagaactctataatgcacccgaggggcgatacaagcaggatgtgtacttgcttcctaagaaaatggatgaatacgttgccagcttgcatctgccatcatttgatgcccaccttacagagctgacagatgaccaagcaaaatatctgggactcaacaaaaatgggccattcaaacctaattattacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggttcctcactctagtcactaaagaaggattttactctcccagcccagaaaggtgattctttctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctattgagtttaattttgtcctctaggatttatttctgttgtccaaaaaaaaaaaaaaagaaaagaaaaattaaggagaatttttggtgttaatgctgaggaattgcttgagtggttagttgttaccaatttctcttttgaacctttggagctaaggatgctgagtctagagaaatgctagtctcaagccctgttaagtccctctgtttctagcccgtagttcatagcatcagtgaactggagccacaacagcaaattctatcagctgtgtaccatacagcttgtgctgaaggcgaatttcttgagccattactcagtataaagcactgagttctatctttaggatttatctttaagagcaaatttctggtcagctgtgcttctgcaacctaaaatatttaaagggaggtaggtgtgggcaggaggaggaatgataaattgggccagggcaagaaaaatctagcttcatataatttgtctgggactatacaccctatataatgttagttttacagaagtaatatgacttttgattgctacataccacaaagagtttatgaactgagatcataaagggcaactgatgtgtgaagaaagtagtcagtacatcctggctcatgctctgaaagaatatccagagaggctctctcaaagatcagggagatgtattcccatgccatgcaccctgcttcccagcatttctgcatggtcaagtgagctttatgctcatgagctttaagtatataattatccaggattttaaatcctcaacttgttctagcttgtgatccctcaaagttgggtcatacgttagtgctagatactagaaattttcacttttccactgatcagagagacagacattaaaaacaaaaatagaagaaaggaaagctttcaccctgcagcttcttagcagggaacaattgtcttgccaaaacttttttcccttttctctcccattttcttttacccaatcccttcttactccttgccagtgtgaccatgctttcttctctgtagatgttaacagttaaggcctattttcctcgggcacttaaccaaccaatcagaacaccacatctgttaggggaggtaacctggccaacagtgtatccatcacgttagccctgctggagggaagggacccacattcacctgccctctgacctgccccttgatcccatatctattaccgtgtccataggaataataggtaagggctctgtctctgtcaagccatgtaacaaaggacactgttaaaaaaaaaaaaaagtctggcatcagagggagcatgtggagagcaacttgggaagaacaagttcattttgtattgaatgatttttaatgaatgcaatattaatccttgcagatgagcaataatcattaaaatcgattaaaatgataagaccttaaaaaa

SEQ ID NO: 10 - protein, 1257 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY

(19) United States
(12) Patent Application Publication
(10) Pub. No.: US 2015/0191791 A1
(43) Pub. Date: Jul. 9, 2015

(54) FGFR2 FUSION GENE

(71) Applicants: NATIONAL CANCER CENTER, Tokyo (JP); LSIP, LLC, Tokyo (JP)

(72) Inventor: Tatsuhiro Shibata, Tokyo (JP)

(73) Assignees: LSIP, LLC, Tokyo (JP); NATIONAL CANCER CENTER, Tokyo (JP)

(21) Appl. No.: 14/412,986

(22) PCT Filed: Jul. 5, 2013
(86) PCT No.: PCT/JP2013/068505

§ 371 Date: Jan. 5, 2015

(30) Foreign Application Priority Data
Jul. 5, 2012 (JP) 2012-151352

(51) Int. Cl. C12Q 1/68; C07K 16/40; C07K 16/28; C12N 9/12; G01N 33/573

(52) U.S. Cl. CPC C12Q 1/6886; C12N 9/12; G01N 33/573; C07K 16/2863; C07K 16/40; C12Y 207/10001; G01N 2333/912; C12Q 2600/158; C12Q 2600/106; G01N 2800/52; C07K 2317/76

(57) ABSTRACT

In order to identify genes that can serve as indicators for predicting the effectiveness of drug treatments in cancers and provide novel methods for predicting the effectiveness of treatments with drugs targeting said genes, transcriptome sequencing was performed of biliary tract cancer. As a result, in-frame fusion transcripts between the FGFR2 gene and other gene (BICC1 or AHCYL1 gene) were identified. It was also found that said gene fusions induce activation of FGFR2 protein, thereby causing canceration of cells. Further, it was demonstrated that the FGFR2 protein activation and canceration caused by said gene fusions can be suppressed by using an FGFR2 inhibitor, and that treatments with an FGFR2 inhibitor are effective in patients with detection of said gene fusions.

### TECHNICAL FIELD

The present invention relates to FGFR2 fusion genes, and more particularly to polynucleotides encoding fusion polypeptides between FGFR2 protein and other protein, polypeptides encoded by said polynucleotides, and a method for detecting said polynucleotides or polypeptides. This invention also relates to a method for determining the effectiveness of cancer treatments with an FGFR2 inhibitor targeting said polynucleotides or polypeptides. This invention further relates to a method for cancer treatment using said effectiveness determination. Furthermore, this invention relates to agents for use in these methods.

### BACKGROUND ART

Biliary tract cancer is a very invasive cancer arising from biliary epithelial cells of intrahepatic biliary tracts (for intrahepatic biliary cancer) and extrahepatic biliary tracts (for extrahepatic biliary cancer). The frequency of this cancer is high mainly in East Asia, but has in recent years been increasing throughout the world including in Europe and the United States. Because of lack of clinical symptoms in the early stage of development, this cancer would in many cases be discovered in advanced stages, and its prognosis is poor. Surgical resection is the only procedure for achieving complete cure, but this cancer has a high rate of recurrence—the 5-year survival rate of operable biliary tract cancer patients is in the range of 15 to 25%. There has not yet been known any chemotherapy that achieves an apparent complete response in inoperable or recurrent patients. However, it has recently been reported that the use of gemcitabine hydrochloride alone or in combination with a platinating agent yields a somewhat more positive outcome than other chemotherapies. Accordingly, there has been a strong need for a new effective therapy for biliary tract cancer, including molecular targeted therapies, and various candidates for the molecular target have heretofore been reported, including EGFR, VEGFR and c-MET.

As driver mutations for biliary tract cancer, KRAS and BRAF mutations have been reported, and the GOPC-ROS1 fusion which had been identified in a brain tumor as a tyrosine kinase fusion gene was also recently reported in biliary tract cancer. No driver mutation has still been identified in patients with no such alterations.

The FGFR (fibroblast growth factor receptor) family is a generic name for transmembrane tyrosine kinase molecules functioning as receptors for FGFs (fibroblast growth factors). There are four known types of FGFR family molecules in humans: FGFRs 1 to 4. The FGFR family contains three immunoglobulin (Ig)-like domains and one transmembrane domain in its extracellular region and a tyrosine kinase domain in its intracellular region. This family is known to contribute to the occurrence and development of many cancers including stomach cancer, breast cancer, uterine cancer, and bladder cancer, and is reported to induce activation in cancers due to various types of genetic alterations (Non-patent Document 1).

For example, as to FGFR1, it is known that gene amplification occurs in breast and ovarian cancers, gene mutation occurs in melanoma, and gene translocation occurs in leukaemia and breast cancer. As to FGFR2, it is known that gene amplification occurs in stomach and breast cancers, and gene mutation occurs in uterine and stomach cancers. As to FGFR3, it is known that gene amplification occurs in bladder and salivary gland cancers, and gene mutation occurs in bladder cancer, uterine cancer, myeloma, and prostate cancer.

The only fusion genes involving FGFR that have heretofore been reported are fusion genes with FGFR1, many of which have been identified in hematological malignancy. Exemplary FGFR1 fusion genes reported in hematological malignancy include FGFR1OP2-FGFR1 (Non-patent Document 2) and ZNF198-FGFR1 (Non-patent Document 3), and the FGFR1 fusion gene reported in solid cancer is FGFR1-ZNF703 in breast cancer (Non-patent Document 4). Regarding aberrant FGFR signaling in biliary tract cancer, expression of the FGFR ligand, FGF, was reported in a literature (Non-patent Document 5).

At present, clinical development is underway of low-molecular-weight inhibitor compounds targeting FGFR, including AZD4547, TKI258, E3810, E7080, BIBF1120, Masitinib, and BGJ398. The development of an antibody therapeutic agent targeting FGFR is also currently in progress (Non-patent Document 1).

On the other hand, BICC1 protein is a molecule having an RNA binding domain and a SAM (sterile alpha motif) domain involved in protein-protein interaction, and negatively regulates Wnt pathway. It was reported that functional abnormalities in this protein cause polycystic kidney disease and pancreas development defects (Non-patent Document 6). Also, AHCYL1 protein is known to have a similar domain to S-adenosyl-L-homocysteine hydrolase and to be involved in secretion in epithelial cells (Non-patent Document 7).

However, the presence of fusion genes involving any of the FGFR2, BICC1 and AHCYL1 genes, and the presence or absence of relationship between such a fusion gene and cancer have yet to be clarified. Also, elucidation of fusion genes and other genes in cancers including biliary tract cancer has not yet been fully achieved. So there is a demand for identifying mutant genes and fusion genes that can serve as indicators for predicting the effectiveness of drug treatments.

### CITATION LIST

### Non-Patent Documents

Non-patent Document 1: Ahmad I., et al., Biochim Biophys Acta., 2012, vol. 1823, p. 850-860

Non-patent Document 2: Popovici C., et al., Blood, 1999, vol. 93, p. 1381-1389

Non-patent Document 3: Xiao S., et al., Nature Genet., 1998, vol. 18, p. 84-87

Non-patent Document 4: “Catalogue of Somatic Mutations in Cancer”

(Translocation; Genes: FGFR1/ZNF703”, [online], last update: 28 Sep. 2011, Wellcome Trust Sanger Institute, U.K., website <URL: http://www.sanger.ac.uk/perl/genetics/CGP/cosmic?action=translocations&id=174&fused=5 9536>

Non-patent Document 5: Ogasawara S., et al., Hepatol Res., 2001, vol. 20, p. 97-113

Non-patent Document 6: Kraus MR., et al., Hum Mutat., 2012, vol. 33, p. 86-90

Non-patent Document 7: Yang D., et al., J Clin Invest., 2011, vol. 121, p. 956-965

### SUMMARY OF INVENTION

### Technical Problem

The present invention has been made in consideration of the above-described problems with the prior art, and has as its object to identify genes that can serve as indicators for predicting the effectiveness of drug treatments in biliary tract cancer and other cancers. Another object of this invention is to provide novel methods for predicting the effectiveness of drug treatments targeting said genes and expression products thereof. Still another object of this invention is to provide methods for treating biliary tract cancer and other cancers on the basis of the prediction of the effectiveness of drug treatments targeting said genes and expression products thereof. Yet another object of this invention is to provide agents for use in detecting said genes and expression products thereof in these methods.

### Solution to Problem

As a result of intensive studies to achieve the above-mentioned objects, the present inventors have identified in-frame fusion transcripts between the FGFR2 gene and other gene (BICC1 gene or AHCYL1 gene) by performing transcriptome sequencing of 8 biliary tract cancer (BTC) specimens. The inventors have found that the fusion gene between the FGFR2 gene and the BICC1 gene (FGFR2-BICC1 fusion gene) is generated by an inversion in chromosome 10, and that the fusion gene between the FGFR2 gene and the AHCYL1 gene (FGFR2-AHCYL1 fusion gene) is generated by a reciprocal translocation between chromosome 1 and chromosome 10. We also have investigated the appearance frequencies of these fusion genes in 102 BTC specimens and, as a result, have found that the appearance frequencies of the FGFR2-AHCYL1 and FGFR2-BICC1 fusion genes are about 6.9% and about 2.0%, respectively.

Further, it is considered that these gene fusions induce activation of FGFR2 protein and hence FGFR2 inhibitors may be therapeutically effective in patients with such activation. Thus, the inventors have introduced each of these fusion genes into normal cells and, as a result, have demonstrated that these cells acquire anchorage-independent colony-forming ability, in other words become cancerous. We also have found that in these cells, the FGFR2 protein kinase is activated and also phosphorylation of its downstream signal, MAPK, is increased.

Further, the inventors have subcutaneously transplanted FGFR2 fusion gene-expressing cells into nude mice and, as a result, have demonstrated that said cells have in vivo tumorigenic ability.

On the other hand, it has also been demonstrated that the activation of FGFR2 protein kinase, the phosphorylation of MAPK, the anchorage-independent colony-forming ability, and the in vivo tumorigenic ability are significantly suppressed by using an FGFR2 inhibitor or through inactivation of the kinase domains of the FGFR2 fusion polypeptide.

On the basis of the above-described findings, the present inventors have found that it is possible to predict the effectiveness of treatments with an FGFR2 inhibitor targeting these gene fusions in biliary tract cancer and other cancers, and that efficient treatments can be performed by administering the inhibitor to patients in whom the treatments with the inhibitor have been determined to be effective on the basis of this prediction; thus, the inventors have completed the present invention.

Therefore, the present invention relates to polynucleotides encoding fusion polypeptides between FGFR2 protein and other protein, polypeptides encoded by said polynucleotides, a method for detecting said polynucleotides or polypeptides, a method for determining the effectiveness of cancer treatments with an FGFR2 inhibitor using the presence of said polynucleotides or polypeptides as an indicator, a method for treatment of cancer utilizing said effectiveness determination, and agents for use in these methods. More specifically, this invention provides the following:

<1> A polynucleotide encoding a polypeptide in which FGFR2 protein and other protein are fused together, wherein the polypeptide is expressed in a cancer cell.

<2> The polynucleotide as set forth in <1>, wherein said other protein is BICC1 protein or AHCYL1 protein.

<3> The polynucleotide as set forth in <1> or <2>, wherein the cancer cell is a biliary tract cancer cell.

<4> A polypeptide encoded by the polynucleotide as set forth in any one of <1> to <3>.

<5> A method for detecting the presence or absence in a sample of the polynucleotide as set forth in any one of <1> to <3> or of the polypeptide as set forth in <4>, the method comprising the steps of:
(a) contacting the sample with an agent intended for specifically detecting the presence or absence of the polynucleotide or the polypeptide in the sample; and
(b) detecting the presence or absence of the polynucleotide or the polypeptide.

<6> An agent for detecting the presence or absence in a sample of the polynucleotide as set forth in any one of <1> to <3> or of the polypeptide as set forth in <4> by the method as set forth in <5>, the agent comprising a polynucleotide or polynucleotides as set forth below in any one of (a) to (c), the polynucleotide or polynucleotides having a chain length of at least 15 nucleotides, or an antibody as set forth below in (d):

(a) a polynucleotide or polynucleotides that are at least one probe selected from the group consisting of a probe that hybridizes to a polynucleotide encoding FGFR2 protein and a probe that hybridizes to a polynucleotide encoding other protein;

(b) a polynucleotide that is a probe that hybridizes to a point of fusion between a polynucleotide encoding FGFR2 protein and a polynucleotide encoding other protein;

(c) polynucleotides that are a pair of primers designed to sandwich a point of fusion between a polynucleotide encoding FGFR2 protein and a polynucleotide encoding other protein; and

(d) an antibody that binds to a polypeptide in which FGFR2 protein and other protein are fused together.

<7> A method for determining the effectiveness of a cancer treatment with an FGFR2 inhibitor, the method comprising the step of detecting the presence or absence in a sample isolated from a patient of the polynucleotide as set forth in any one of <1> to <3> or of the polypeptide as set forth in <4>, wherein in a case where the presence of the polynucleotide or the polypeptide is detected, the cancer treatment with the FGFR2 inhibitor is determined to be highly effective in the patient.

<8> An agent for determining the effectiveness of a cancer treatment with an FGFR2 inhibitor by the method as set forth in <7>, the agent comprising a polynucleotide or polynucleotides as set forth below in any one of (a) to (c), the polynucleotide or polynucleotides having a chain length of at least 15 nucleotides, or an antibody as set forth below in (d):

(a) a polynucleotide or polynucleotides that are at least one probe selected from the group consisting of a probe that hybridizes to a polynucleotide encoding FGFR2 protein and a probe that hybridizes to a polynucleotide encoding other protein;

(b) a polynucleotide that is a probe that hybridizes to a point of fusion between a polynucleotide encoding FGFR2 protein and a polynucleotide encoding other protein;

(c) polynucleotides that are a pair of primers designed to sandwich a point of fusion between a polynucleotide encoding FGFR2 protein and a polynucleotide encoding other protein; and

(d) an antibody that binds to a polypeptide in which FGFR2 protein and other protein are fused together.

<9> A method for treatment of cancer, comprising the step of administering an FGFR2 inhibitor to a patient in whom a cancer treatment with the FGFR2 inhibitor has been determined to be highly effective by the method as set forth in <7>.

<10> A therapeutic agent for cancer, comprising an FGFR2 inhibitor as an active ingredient, the agent which is to be administered to a patient in whom a cancer treatment with the FGFR2 inhibitor has been determined to be highly effective by the method as set forth in <7>.

### Advantageous Effects of Invention

The present invention enables effective detection of fusion genes between the FGFR2 gene and other gene, and expression products thereof. This invention also makes it possible to predict the effectiveness of various treatments on cancers, in particular the effectiveness of cancer treatments with an FGFR2 inhibitor, on the basis of the detection of said fusion genes and expression products thereof. This prediction makes it possible to avoid administration of a drug to cancer patients conceivably not responsive to the administration of the drug, thereby allowing efficient cancer treatments.

### BRIEF DESCRIPTION OF DRAWINGS

FIG. 1 is a schematic diagram showing the fusion between the FGFR2 gene and the BICC1 gene generated by an inversion in human chromosome 10.

FIG. 2 is a schematic diagram showing the fusion between FGFR2 protein and BICC1 protein. More specifically, this schematic diagram shows that the extra-membranous domains (IgI to IgIII), transmembrane (TM) domain and kinase domains of FGFR2 protein are fused with the C-terminal moiety of BICC1 protein. This diagram also shows that a polynucleotide encoding the entire FGFR2 fusion polypeptide including the FGFR2 kinase region can be detected by using RT-PCR primers each hybridizing to exon 16 or 18 in the kinase region of the FGFR2 gene and to exon 7/8 or 21 in the BICC1 gene.

FIG. 3 is a schematic diagram showing the fusion between the FGFR2 gene and the AHCYL1 gene generated by a balanced translocation (reciprocal translation) between human chromosome 1 and human chromosome 10.

FIG. 4 is a schematic diagram showing the fusion between FGFR2 protein and AHCYL1 protein. More specifically, this schematic diagram shows that the extra-membranous domains (IgI to IgIII), transmembrane (TM) domain and kinase domains of FGFR2 protein are fused with the C-terminal moiety of AHCYL1 protein. This diagram also shows that a polynucleotide encoding the entire FGFR2 fusion polypeptide including the FGFR2 kinase region can be detected by using RT-PCR primers each hybridizing to exon 16 or 18 in the kinase region of the FGFR2 gene and to exon 9 or 20 in the AHCYL1 gene.

FIG. 5 is a schematic diagram showing a method for detecting the inventive fusion gene by the FISH method. More specifically, this schematic diagram shows that in the case of the FGFR2-BICC1 fusion gene, the presence of the FGFR2-BICC1 fusion gene can be detected by designing FISH probes each specific for a portion toward the 5′ end of the FGFR2 gene or for a portion toward the 3′ end of the BICC1 gene.

FIG. 6 is a schematic diagram showing a method for detecting the inventive fusion gene by the FISH method. More specifically, this schematic diagram shows that in the case of the FGFR2-AHCYL1 fusion gene, the presence of the FGFR2-AHCYL1 fusion gene can be detected by designing FISH probes each specific for a portion toward the 5′ end of the FGFR2 gene or for a portion toward the 3′ end of the AHCYL1 gene.

FIG. 7 is a set of photos showing the results of observing, under a microscope, the anchorage-independent colony formation in normal murine fibroblast lines each stably expressing the wild-type or the mutated FGFR2 fusion polypeptide, which were each seeded in a soft agar medium. In this figure, the “FGFR2-AHCYL1” panel shows the result for a cell line expressing the wild-type FGFR2-AHCYL1 fusion polypeptide, the “FGFR2-AHCYL1-KD” panel shows the result for a cell line expressing the mutated FGFR2-AHCYL1 fusion polypeptide, the “FGFR2-BICC1” panel shows the result for a cell line expressing the wild-type FGFR2-BICC1 fusion polypeptide, and the “FGFR2-BICC1-KD” panel shows the result for a cell line expressing the mutated FGFR2-BICC1 fusion polypeptide. The bars in these panels indicate the length of 100 μm.

FIG. 8 is a graph showing the results of analyzing the anchorage-independent colony-forming ability of normal murine fibroblast lines each stably expressing the wild-type or the mutated FGFR2 fusion polypeptide, which were each seeded in a soft agar medium. In this graph, the vertical axis represents a relative value of the number of colonies formed in each of the cell lines expressing either one of the mutated FGFR2 fusion polypeptides, with respect to that number in each of the corresponding cell lines expressing either one of the wild-type FGFR2 fusion polypeptides, which is taken as 100. An asterisk shows that a significant difference was observed (the p-value is 0.05 or less).

FIG. 9 is a graph showing the results of analyzing the anchorage-independent colony-forming ability of normal murine fibroblast lines stably expressing either one of the wild-type FGFR2 fusion polypeptides and a normal murine fibroblast line stably expressing the EZR-ROS1 fusion polypeptide, which were each seeded in a soft agar supplemented with an FGFR kinase inhibitor (BGJ398 or PD173074). In this graph, the “BGJ” bars show the results of colony formation in the presence of BGJ398 (at a BGJ398 concentration in medium of 0.2 nmol/mL), the “PD” bars show the results of colony formation in the presence of PD173074 (at a PD173074 concentration in medium of 0.2 nmol/mL), and the “Blank” bars show the results of colony formation in the absence of an FGFR kinase inhibitor. The vertical axis represents a relative value of the number of colonies with respect to the number of colonies formed in each of the cell lines expressing any one of said fusion polypeptides in the absence of an FGFR kinase inhibitor, which is taken as 100. An asterisk shows that a significant difference was observed (the p-value is 0.05 or less).

FIG. 10 is a set of photos showing the results of analyzing by Western blotting the phosphorylation of various proteins in a normal murine fibroblast line stably expressing the wild-type FGFR2-AHCYL1 fusion polypeptide (“FGFR2-AH”) or a normal murine fibroblast line stably expressing the EZR-ROS1 fusion polypeptide (“EZR-ROS1”), which were subjected to serum starvation and then cultured in the presence of an FGFR kinase inhibitor (BGJ398 or PD173074). In this figure, the 1st and 6th lanes from the left show the results of culturing in the absence of an FGFR kinase inhibitor; the 2nd and 7th lanes from the left show the results of culturing in the presence of BGJ398 (at a BGJ398 concentration in medium of 0.2 nmol/mL); the 3rd and 8th lanes from the left show the results of culturing in the presence of BGJ398 (at a BGJ398 concentration in medium of 1.0 nmol/mL); the 4th and 9th lanes from the left show the results of culturing in the presence of PD173074 (at a PD173074 concentration in medium of 0.2 nmol/mL); and the 5th and 10th lanes from the left show the results of culturing in the presence of PD173074 (at a PD173074 concentration of 1.0 nmol/mL). Since these fusion polypeptides further have the FLAG tag bound thereto and express it intracellularly, the “FLAG tag” row in this figure shows the results of detection of the respective fusion polypeptides through this tag. The “p-FGFR (Y653/Y654)” row shows the results of detection of phosphorylated FGFR. The “STAT3” and “p-STAT3 (Y705)” rows show the results of detection of STAT3 and phosphorylated STAT3, respectively. The “AKT” and “p-AKT (S473)” show the results of detection of AKT and phosphorylated AKT, respectively. The “MAPK” and “p-MAPK (T202/Y204)” rows show the results of detection of MAPK and phosphorylated MAPK, respectively. The “β-actin” row shows that the amount of protein used as an internal standard is uniform among the lanes.

FIG. 11 is a set of photos showing the results of subcutaneously transplanting each of normal murine fibroblast lines expressing the wild-type or the mutated FGFR2 fusion polypeptide into immunodeficient mice. In this figure, the “Wild-type” panels are photos taken upon the observation of the immunodeficient mice 18 days after the mice were transplanted subcutaneously with any of the cell lines expressing either one of the wild-type FGFR2 fusion polypeptides. Triangles indicate formed tumors. “8/8” represents that each type of said cells was transplanted into two sites of each of four mice, a total of eight sites, and as a result, tumorigenesis was observed in all of the eight sites. The “mutated” panels are photos taken upon the observation of the immunodeficient mice 18 days after the mice were transplanted subcutaneously with each of the cell lines expressing either one of the mutated FGFR2 fusion polypeptides. “0/6” represents that each type of said cells was transplanted into two sites of each of three mice, a total of six sites, and as a result, no tumorigenesis was observed in any of the six sites.

### DESCRIPTION OF EMBODIMENTS

<Polynucleotides Encoding Fusion Polypeptides between FGFR2 Protein and Other Protein, and Polypeptides Encoded by the Polynucleotides>

As disclosed below in Examples, multiple cases of fusion between the FGFR2 gene and other gene were first discovered according to the present invention. Therefore, this invention provides a polynucleotide encoding a fusion polypeptide between FGFR2 protein and other protein (said polynucleotide and said fusion polypeptide are to be hereinafter also referred to as the “FGFR2 fusion polynucleotide” and the “FGFR2 fusion polypeptide”, respectively).

The “FGFR2 fusion polypeptide” as referred to in the present invention means a polypeptide in which the full length or part of FGFR2 protein is fused with the full length or part of other protein. Also, the “FGFR2 fusion polynucleotide” as referred to in this invention means a polynucleotide in which a polynucleotide encoding the full length or part of FGFR2 protein is fused with a polynucleotide encoding the full length or part of other protein.

Examples of the FGFR2 fusion polynucleotide include: a polynucleotide encoding a fusion polypeptide between FGFR2 protein and BICC1 protein (said polynucleotide and said fusion polypeptide are to be hereinafter also referred to as the “FGFR2-BICC1 fusion polynucleotide” and the “FGFR2-BICC1 fusion polypeptide”, respectively); and a polynucleotide encoding a fusion polypeptide between FGFR2 protein and AHCYL1 protein (said polynucleotide and said fusion polypeptide are to be hereinafter also referred to as the “FGFR2-AHCYL1 fusion polynucleotide” and the “FGFR2-AHCYL1 fusion polypeptide”, respectively).

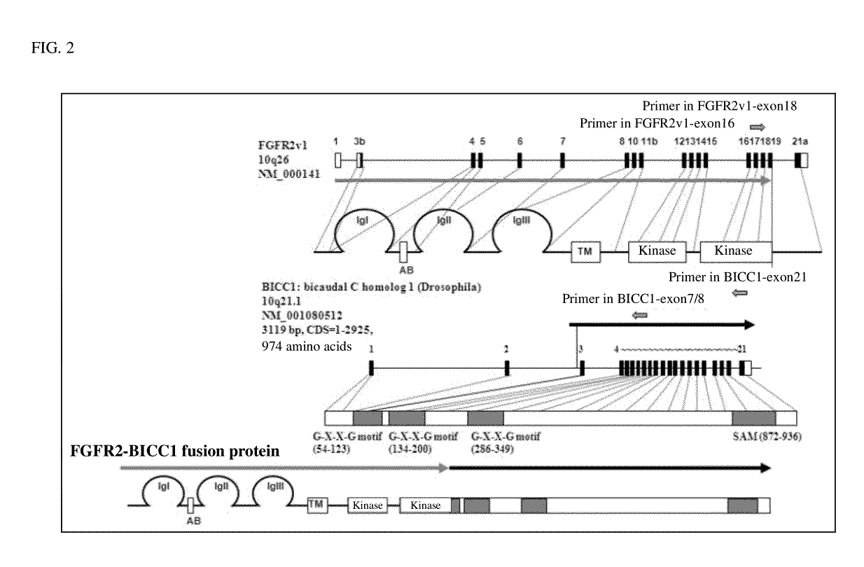

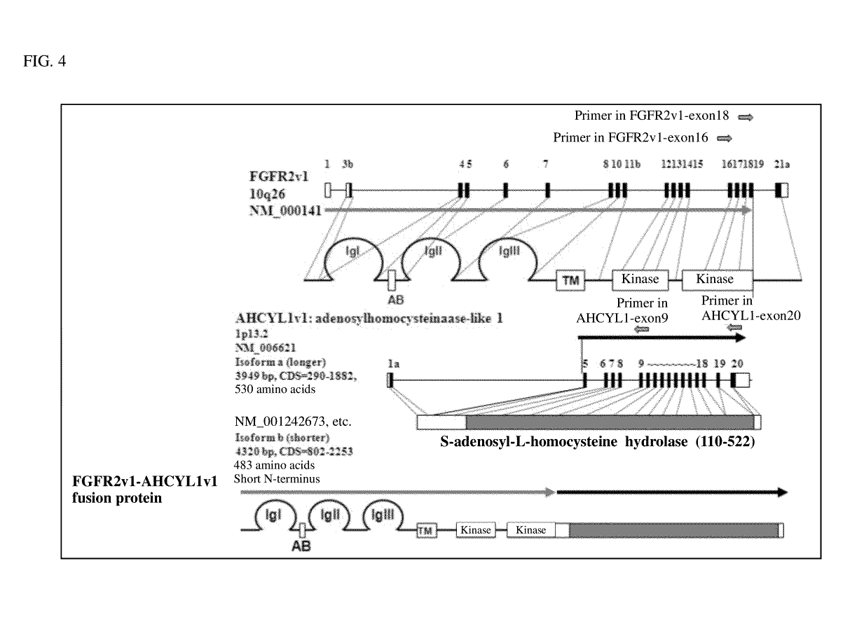

The “FGFR2 (fibroblast growth factor receptor 2) protein” according to the present invention refers to a protein encoded by the gene located at a long arm of chromosome 10 (10q26) in humans. In this invention, the “FGFR2 protein”, as far as it is derived from humans, can be exemplified by: the protein identified by RefSeq ID: NP_{—}000132 (isoform 1); the protein identified by RefSeq ID: NP_{—}075259 (isoform 2); the protein identified by RefSeq ID: NP_{—}001138385 (isoform 3); the protein identified by RefSeq ID: NP_{—}001138386 (isoform 4); the protein identified by RefSeq ID: NP_{—}001138387 (isoform 5); the protein identified by RefSeq ID: NP_{—}001138388 (isoform 6); the protein identified by RefSeq ID: NP_{—}001138389 (isoform 7); the protein identified by RefSeq ID: NP_{—}001138390 (isoform 8); and the protein identified by RefSeq ID: NP_{—}001138391 (isoform 9). These FGFR2 protein isoforms are slightly different from each other in terms of extra-membranous domain structure (e.g., the presence or absence of IgI; refer to FIGS. 2 and 4). In this invention, the “FGFR2 protein”, as far as it is derived from humans, is typified by the protein consisting of the amino acid sequence of SEQ ID NO: 2 (isoform 1). The polynucleotide encoding the FGFR2 protein is typified by the polynucleotide consisting of the nucleotide sequence of SEQ ID NO: 1.

The “BICC1 (bicaudal C homolog 1 (Drosophila)) protein” according to the present invention refers to a protein encoded by the gene located at a long arm of chromosome 10 (10q21.1) in humans. In this invention, the “BICC1 protein”, as far as it is derived from humans, is typified by the protein consisting of the amino acid sequence of SEQ ID NO: 4. The polynucleotide encoding the BICC1 protein is typified by the polynucleotide consisting of the nucleotide sequence of SEQ ID NO: 3.

The “AHCYL1 (S-adenosyl-L-homocysteine hydrolase-like 1; AHCY-like 1) protein” according to the present invention is also referred to as IRBIT (inositol 1,4,5-trisphosphate receptor 1 (ITPR1)-binding protein released with inositol 1,4,5-trisphosphate, IRBIT, ITPR1-binding protein released with IP3) or DCAL (dendritic cell-expressed AHCY-like protein), and means a protein encoded by the gene located at a short arm of chromosome 1 (1p13.2) in humans. In this invention, the “AHCYL1 protein”, as far as it is derived from humans, can be exemplified by: the protein identified by RefSeq ID: NP_{—}006612 (isoform a); and the protein identified by RefSeq ID: NP_{—}001229603 (isoform b). AHCYL1 protein isoform a is characterized by having a longer N-terminus than isoform b. In this invention, the “AHCYL1 protein”, as far as it is derived from humans, is typified by the protein consisting of the amino acid sequence of SEQ ID NO: 6 (isoform a). The polynucleotide encoding the AHCYL1 protein is typified by the polynucleotide consisting of the nucleotide sequence of SEQ ID NO: 5.

The FGFR2 fusion polynucleotide is typified by a polynucleotide encoding a polypeptide in which the N-terminal moiety of FGFR2 protein is fused with the C-terminal moiety of other protein.

In the present invention, the “N-terminal moiety of FGFR2 protein” is typified by a moiety comprising the extra-membranous domains, transmembrane domain, and kinase domains of the FGFR2 protein (refer to FIGS. 2 and 4).

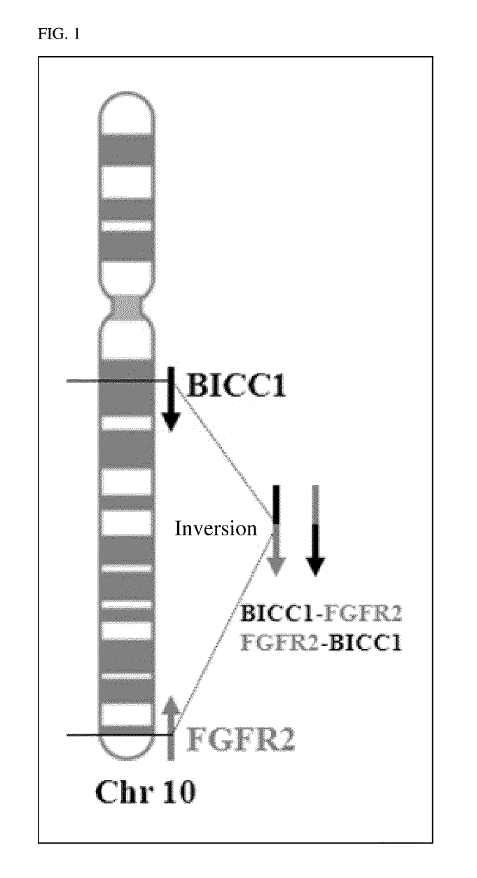

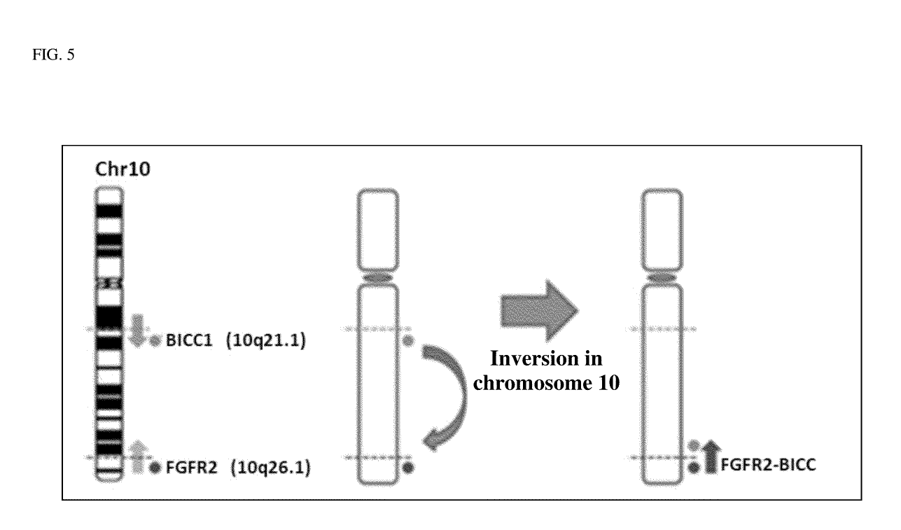

For the purpose of the present invention, the “polynucleotide encoding a polypeptide in which the N-terminal moiety of FGFR2 protein is fused with the C-terminal moiety of BICC1 protein” is typified by a polynucleotide generated by an inversion in chromosome 10, as shown in FIGS. 1 and 5. More specifically, this term refers to a polynucleotide encoding a polypeptide in which a polypeptide region (extra-membranous domains, transmembrane domain, and kinase domains) of the FGFR2 protein that is encoded by exons 1-19 is fused with a polypeptide region of the BICC1 protein that is encoded by exons 3-21 (e.g., the polypeptide consisting of the amino acid sequence of SEQ ID NO: 8). Such a polynucleotide is exemplified by the polynucleotide consisting of the nucleotide sequence of SEQ ID NO: 7.

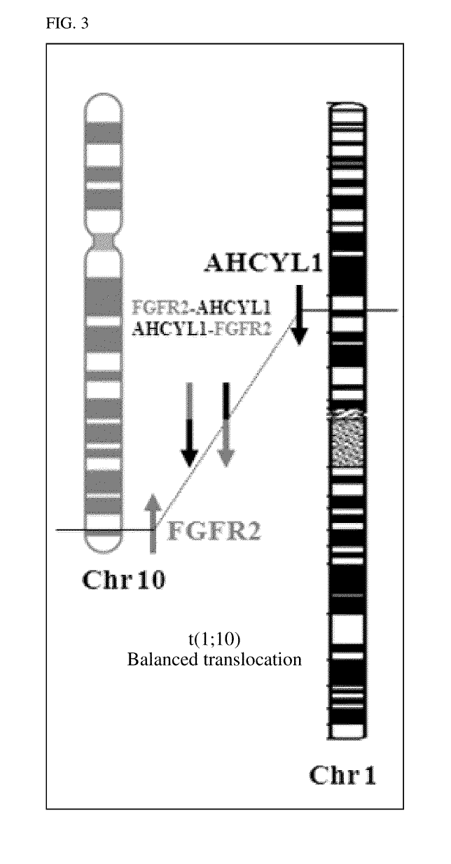

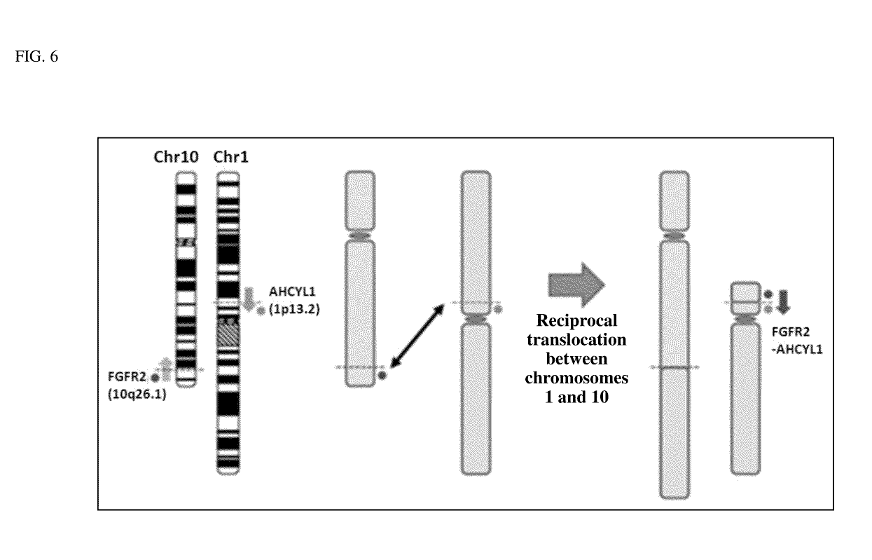

For the purpose of the present invention, the “polynucleotide encoding a polypeptide in which the N-terminal moiety of FGFR2 protein is fused with the C-terminal moiety of AHCYL1 protein” is typified by a polynucleotide generated by a reciprocal translocation between chromosome 1 and chromosome 10, as shown in FIGS. 3 and 6. More specifically, this term refers to a polynucleotide encoding a polypeptide in which a polypeptide region (extra-membranous domains, transmembrane domain, and kinase domains) of the FGFR2 protein that is encoded by exons 1-19 are fused with a polypeptide region of the AHCYL1 protein that is encoded by exons 5-20 (e.g., the polypeptide consisting of the amino acid sequence of SEQ ID NO: 10). Such a polynucleotide is exemplified by the polynucleotide consisting of the nucleotide sequence of SEQ ID NO: 9.

In the present invention, the amino acid sequences of “FGFR2 protein”, “BICC1 protein” and “AHCYL1 protein”, and the nucleotide sequences of the genes encoding said proteins can mutate in nature (i.e., in a non-artificial way). Thus, the amino acid sequence of the “FGFR2 fusion polypeptide” and the nucleotide sequence of the “FGFR2 fusion polynucleotide” can also mutate in nature (i.e., in a non-artificial way). Said amino acid sequences and nucleotide sequences may be artificially modified. Such mutants are also encompassed by this invention.

Certain exemplary mutants of the FGFR2-BICC1 fusion polypeptide include proteins consisting of an amino acid sequence derived from the amino acid sequence of SEQ ID NO: 8 by substitution, deletion, addition and/or insertion of one or more amino acids. Certain exemplary mutants of the FGFR2-AHCYL1 fusion polypeptide include proteins consisting of an amino acid sequence derived from the amino acid sequence of SEQ ID NO: 10 by substitution, deletion, addition and/or insertion of one or more amino acids.

As used herein, the term “more” refers to generally 50 or fewer amino acids, preferably 30 or fewer amino acids, more preferably 10 or fewer amino acids, and particularly preferably several or fewer amino acids (for example, five or fewer amino acids, three or fewer amino acids, two or one amino acid, one amino acid).

Other exemplary mutants of the FGFR2-BICC1 fusion polypeptide include polypeptides encoded by a DNA that hybridizes under stringent conditions to a DNA consisting of the nucleotide sequence of SEQ ID NO: 7. Other exemplary mutants of the FGFR2-AHCYL1 fusion polypeptide include polypeptides encoded by a DNA that hybridizes under stringent conditions to a DNA consisting of the nucleotide sequence of SEQ ID NO: 9.

Exemplary high stringent hybridization conditions are 0.2×SSC at 65° C., and exemplary low stringent hybridization conditions are 2.0×SSC at 50° C.

Still other exemplary mutants of the FGFR2-BICC1 fusion polypeptide include polypeptides consisting of an amino acid sequence having at least 80% (for example, at least 85%, 90%, 95%, 97%, 99%) homology to the amino acid sequence of SEQ ID NO: 8. Still other exemplary mutants of the FGFR2-AHCYL1 fusion polypeptide include polypeptides consisting of an amino acid sequence having at least 80% (for example, at least 85%, 90%, 95%, 97%, 99%) homology to the amino acid sequence of SEQ ID NO: 10.

Sequence homology can be determined using the BLASTP (amino acid level) program (Altschul, et al., J. Mol. Biol., 1990, 215: 403-410). This program is based on the algorithm BLAST developed by Karlin and Altschul (Proc. Natl. Acad. Sci. USA, 1990, 87: 2264-2268; and Proc. Natl. Acad. Sci. USA, 1993, 90: 5873-5877). When amino acid sequence analysis is performed using BLASTP, the parameters are typically set as follows: score=50 and wordlength=3. Amino acid sequence analysis using the Gapped BLAST program can be performed as per the descriptions in Altschul, et al. (Nucleic Acids Res., 1997, 25: 3389-3402). When amino acid sequence analysis is performed using the BLAST and Gapped BLAST programs, the default parameters of these programs are used. The specific procedures for conducting these analyses are known.

Exemplary mutants of the FGFR2-BICC1 fusion polynucleotide include polynucleotides encoding the above-mentioned mutants of the FGFR2-BICC1 fusion polypeptide, and polynucleotides encoding degenerate variants of said polypeptide which have no amino acid mutation. Exemplary mutants of the FGFR2-AHCYL1 fusion polynucleotide include polynucleotides encoding the above-mentioned mutants of the FGFR2-AHCYL1 fusion polypeptide, and polynucleotides encoding degenerate variants of said polypeptide which have no amino acid mutation.

Exemplary forms of the “FGFR2 fusion polynucleotide” according to the present invention include mRNA, cDNA, and genomic DNA. It is possible for those skilled in the art using a known hybridization technique to isolate the “FGFR2 fusion polynucleotide” from a cDNA library or genomic DNA library prepared from BTC or other cancers that harbor a fusion gene between the FGFR2 gene and other gene. The polynucleotide can also be prepared by amplification utilizing a known gene amplification technique (PCR), with the mRNA, cDNA or genomic DNA prepared from BTC or other cancers being used as a template.

Furthermore, after the thus-prepared polynucleotide is inserted into an appropriate expression vector, the vector is introduced into a cell-free protein synthesis system (e.g., reticulocyte extract, wheat germ extract) and the system is incubated, or alternatively the vector is introduced into appropriate cells (e.g., E coli., yeast, insect cells, animal cells) and the resulting transformant is cultured; in either way, the FGFR2 fusion polypeptide can be prepared.

As mentioned above, the “FGFR2 fusion polypeptide” and “FGFR2 fusion polynucleotide” according to the present invention encompasses, in a broad sense, both those having naturally occurring sequences (including those mutated in nature) and those having artificially modified sequences. However, it should be noted that the “FGFR2 fusion polypeptide” and “FGFR2 fusion polynucleotide”, particularly if these terms are used as an object of the detection as described below, mainly refer to those having naturally occurring sequences (including those mutated in nature).

<Method for Detecting the Presence or Absence of the FGFR2 Fusion Polypeptides or the FGFR2 Fusion Polynucleotides>

The present invention also provides a method for detecting the presence or absence of the FGFR2 fusion polynucleotides or the FGFR2 fusion polypeptides in a sample. The detection method of this invention comprises the steps of: (a) contacting the sample with an agent intended for specifically detecting the presence or absence of the polynucleotide or the polypeptide in the sample; and (b) detecting the presence or absence of the polynucleotide or the polypeptide.

For the purpose of the present invention, the term “sample” includes not only biological samples (for example, cells, tissues, organs, body fluids (e.g., blood, lymphs), digestive juices, sputum, bronchoalveolar/bronchial lavage fluids, urine, and feces), but also nucleic acid extracts from these biological samples (for example, genomic DNA extracts, mRNA extracts, and cDNA and cRNA preparations from mRNA extracts) and protein extracts. The sample may also be the one that is fixed with formalin or alcohol, frozen, or embedded in paraffin.

Further, the genomic DNA, mRNA, cDNA or protein can be prepared by those skilled in the art through considering various factors including the type and state of the sample and selecting a known technique suitable therefor.

In the present invention, the “detection of the presence or absence of the FGFR2 fusion polynucleotide or the FGFR2 fusion polypeptide” can be performed on genomic DNAs encoding said fusion polypeptide, transcripts from said genomic DNAs, or translation products from said transcripts.

Since a genomic DNA encoding the FGFR2-BICC1 fusion polypeptide is formed by an inversion in chromosome 10, the “detection of the presence or absence of the FGFR2-BICC1 fusion polynucleotide” may be achieved by detecting this phenomenon of inversion (refer to FIGS. 1 and 5). The detection of such an inversion may be achieved, for example, by detecting a split between the portion consisting of the exon 19-coding region of the FGFR2 gene and a region upstream from said coding region toward the 5′ end, and the portion consisting of the exon 20-coding region of the FGFR2 gene and a region downstream from said coding region toward the 3′ end, or by detecting a split between the portion consisting of the exon 2-coding region of the BICC1 gene and a region upstream from said coding region toward the 5′ end, and the portion consisting of the exon 3-coding region of the BICC1 gene and a region downstream from said coding region toward the 3′ end.

Since a genomic DNA encoding the FGFR2-AHCYL1 fusion polypeptide is formed by a reciprocal translocation between chromosome 1 and chromosome 10, the “detection of the presence or absence of the FGFR2-AHCYL1 fusion polynucleotide” may be achieved by detecting this phenomenon of reciprocal translocation (refer to FIGS. 3 and 6). The detection of such a reciprocal translocation may be achieved, for example, by detecting a split between the portion consisting of the exon 19-coding region of the FGFR2 gene and a region upstream from said coding region toward the 5′ end, and the portion consisting of the exon 20-coding region of the FGFR2 gene and a region downstream from said coding region toward the 3′ end, or by detecting a split between the portion consisting of the exon la-coding region of the AHCYL1 gene and a region upstream from said coding region toward the 5′ end, and the portion consisting of the exon 5-coding region of the AHCYL1 gene and a region downstream from said coding region toward the 3′ end.

The “detection of the presence of absence of the FGFR2 fusion polynucleotide” according to the present invention can be performed using a known method. Exemplary known methods that can be used in the detection on the “genomic DNAs encoding said fusion polypeptide” include in situ hybridization (ISH) using fluorescence or other means, genomic PCR, direct sequencing, Southern blotting, and genome microarray analysis. Exemplary known methods that can be used in the detection on the “transcripts from said genomic DNAs” include RT-PCR, direct sequencing, Northern blotting, dot blotting, and cDNA microarray analysis.

According to in situ hybridization, genomic DNAs encoding the FGFR2 fusion polypeptide can be detected by contacting a biological sample with the polynucleotide or polynucleotides noted below in (a) or (b), which have a chain length of at least 15 nucleotides:

(a) a polynucleotide or polynucleotides that are at least one probe selected from the group consisting of a probe that hybridizes to a polynucleotide encoding FGFR2 protein and a probe that hybridizes to a polynucleotide encoding other protein; or

(b) a polynucleotide that is a probe that hybridizes to a point of fusion between a polynucleotide encoding FGFR2 protein and a polynucleotide encoding other protein.

In relation to the detection of genomic DNAs encoding the FGFR2 fusion polypeptide, the “polynucleotide encoding FGFR2 protein” according to the present invention, as far as it is derived from humans, is typified by the gene consisting of the DNA sequence of positions 1 to 120129 in the genome sequence identified by RefSeq ID: NG_{—}012449 (“FGFR2 gene”).

The “polynucleotide encoding BICC1 protein” according to the present invention, as far as it is derived from humans, is typified by the gene consisting of the DNA sequence of positions 1 to 315942 in the genome sequence identified by RefSeq ID: NG_{—}029759 (“BICC1 gene”).

The “polynucleotide encoding AHCYL1 protein” according to the present invention, as far as it is derived from humans, is typified by the gene consisting of the DNA sequence of positions 1 to 38979 in the genome sequence identified by RefSeq ID: NG_{—}029182 (“AHCYL1 gene”).

However, the DNA sequences of the genes can vary in nature (i.e., in a non-artificial way) due to their mutations and the like. Thus, such naturally occurring mutants can also be encompassed by the present invention (the same applies hereinafter).

The polynucleotide(s) of (a) or (b) according to the present invention has a length of at least 15 nucleotides, preferably at least 20 nucleotides, more preferably from 100 to 1000 nucleotides.

The polynucleotide(s) of (a) according to the present invention can be of any type as far as it is capable of detecting the presence of a genomic DNA encoding the FGFR2 fusion polypeptide in the foregoing biological sample by hybridizing to a nucleotide sequence targeted by said polynucleotide, or more specifically to a polynucleotide encoding FGFR2 protein or a polynucleotide encoding other protein. The polynucleotide(s) of (a) is preferably any of the polynucleotides noted below in (a1) to (a5):

(a1) a combination of a polynucleotide that hybridizes to the portion consisting of the exon 19-coding region of the FGFR2 gene and a region upstream from said coding region toward the 5′ end (this polynucleotide is to be hereinafter also referred to as “5′ FGFR2 probe”), and a polynucleotide that hybridizes to the portion consisting of the exon 3-coding region of the BICC1 gene and a region downstream from said coding region toward the 3′ end (this polynucleotide is to be hereinafter also referred to as “3′ BICC1 probe”);

(a2) a combination of 5′ FGFR2 probe and a polynucleotide that hybridizes to the portion consisting of the exon 5-coding region of the AHCYL1 gene and a region downstream from said coding region toward the 3′ end (this polynucleotide is to be hereinafter also referred to as “3′ AHCYL1 probe”);

(a3) a combination of 5′ FGFR2 probe and a polynucleotide that hybridizes to the portion consisting of the exon 20-coding region of the FGFR2 gene and a region downstream from said coding region toward the 3′ end (this polynucleotide is to be hereinafter also referred to as “3′ FGFR2 probe”);

(a4) a combination of a polynucleotide that hybridizes to the portion consisting of the exon 2-coding region of the BICC1 gene and a region upstream from said coding region toward the 5′ end (this polynucleotide is to be hereinafter also referred to as “5′ BICC1 probe”), and 3′ BICC1 probe; and

(a5) a combination of a polynucleotide that hybridizes to the portion consisting of the exon 1a-coding region of the AHCYL1 gene and a region upstream from said coding region toward the 5′ end (this polynucleotide is to be hereinafter also referred to as “5′ AHCYL1 probe”), and 3′ AHCYL1 probe.

In the present invention, it is preferred from the viewpoints of specificity for a target nucleotide sequence and detection sensitivity that the region to which the pair of polynucleotides of (a1) or (a2) as used for in situ hybridization is to hybridize (such a region is to be hereinafter referred to as the “target nucleotide sequence”) should be a region extending for not more than 1000000 nucleotides from a point of fusion between a polynucleotide encoding FGFR2 protein and a polynucleotide encoding other protein. And it is preferred from the viewpoints of the same factors that the region to which the pair of polynucleotides of (a3), (a4) or (a5) as used for in situ hybridization is to hybridize should be a region extending for not more than 1000000 nucleotides from a breakpoint in a polynucleotide encoding FGFR2 protein, in a polynucleotide encoding BICC1 protein, or in a polynucleotide encoding AHCYL1 protein.

In the present invention, the polynucleotide of (b) as used for in situ hybridization can be of any type as far as it is capable of detecting the presence of a genomic DNA encoding the FGFR2 fusion polypeptide in the foregoing biological sample by hybridizing to a nucleotide sequence targeted by said polynucleotide, more specifically to a point of fusion between a polynucleotide encoding FGFR2 protein and a polynucleotide encoding other protein. Typical examples of the polynucleotide of (b) are those which each hybridize to a genomic DNA encoding a polynucleotide consisting of the nucleotide sequence of SEQ ID NO: 7 or 9, for example, those which each hybridize to a point of fusion between a polynucleotide encoding FGFR2 protein and a polynucleotide encoding other protein.

Further, in the present invention, it is preferred from the viewpoints of further improvement of specificity for a target nucleotide sequence and detection sensitivity that the polynucleotide or polynucleotides of (a) or (b) as used for in situ hybridization be a group consisting of multiple types of polynucleotides which can cover the entire target nucleotide sequence. In such a case, the polynucleotides constituting said group each have a length of at least 15 nucleotides, preferably at least 20 nucleotides, more preferably from 100 to 1000 nucleotides.

The polynucleotide or polynucleotides of (a) or (b) as used for in situ hybridization are preferably labeled for detection with a fluorescent dye or other means. Examples of such a fluorescent dye include, but are not limited to, DEAC, FITC, R6G, TexRed, and Cy5. Aside from fluorescent dyes, the polynucleotide may also be labeled with a dye (chromogen) such as DAB or with silver or other means based on enzymatic metal deposition.

In the process of in situ hybridization, a probe for a polynucleotide encoding FGFR2 protein and a probe for a polynucleotide encoding other protein are preferably each labeled with a different dye. If, as the result of in situ hybridization using the probe combination of (a1) or (a2) which consists of probes labeled with different dyes, an overlap is observed between signals emitted from labels on these probes, then it can be determined that a genomic DNA encoding the FGFR2 fusion polypeptide has been detected successfully. Also, if, as the result of in situ hybridization using the probe combination of (a3), (a4) or (a5) which consists of probes labeled with different dyes, a split is observed between signals emitted from labels on these probes, then it can be determined that a genomic DNA encoding the FGFR2 fusion polypeptide has been detected successfully.

Polynucleotide labeling can be effected by a known method. For example, the polynucleotides can be labeled by nick translation or random priming, in which the polynucleotides are caused to incorporate substrate nucleotides labeled with a fluorescent dye or other means.

The conditions for contacting the foregoing biological sample with the polynucleotide(s) of (a) or (b) in the process of in situ hybridization can vary with various factors including the length of said polynucleotide(s); and exemplary high stringent hybridization conditions are 0.2×SSC at 65° C., and exemplary low stringent hybridization conditions are 2.0×SSC at 50° C. Those skilled in the art could realize comparable stringent hybridization conditions to those mentioned above, by appropriately selecting salt concentration (e.g., SSC dilution rate), temperature, and various other conditions including concentrations of surfactant (e.g., NP-40) and formamide, and pH.

In addition to in situ hybridization, other examples of the method for detecting a genomic DNA encoding the FGFR2 fusion polypeptide using the polynucleotide(s) of (a) or (b) include Southern blotting, Northern blotting and dot blotting. According to these methods, the fusion gene is detected by hybridizing the polynucleotide(s) of (a) or (b) to a membrane in which a nucleic acid extract from the foregoing biological sample is transcribed. In the case of using the polynucleotide(s) of (a), if a polynucleotide that hybridizes to a polynucleotide encoding FGFR2 protein and a polynucleotide that hybridizes to a polynucleotide encoding other protein recognize the same band present in the membrane, then it can be determined that a genomic DNA encoding the FGFR2 fusion polypeptide has been detected successfully.

Additional examples of the method for detecting a genomic DNA encoding the FGFR2 fusion polypeptide using the polynucleotide of (b) include genome microarray analysis and DNA microarray analysis. According to these methods, the genomic DNA is detected by preparing an array in which the polynucleotide of (b) is immobilized on a substrate and bringing the foregoing biological sample into contact with the polynucleotide immobilized on the array.

In the process of PCR or sequencing, the polynucleotides noted below in (c) can be used to specifically amplify part or all of the FGFR2 fusion polynucleotide using a DNA (genomic DNA, cDNA) or RNA prepared from the foregoing biological sample as a template:

(c) polynucleotides that are a pair of primers designed to sandwich a point of fusion between a polynucleotide encoding FGFR2 protein and a polynucleotide encoding other protein.

The “polynucleotides that are a pair of primers” refers to a primer set designed such that in the foregoing fusion polynucleotide or the like to be targeted, one of the primers hybridizes to a region of the FGFR2 gene and the other primer hybridizes to a region of other gene. These polynucleotides have a length of generally 15-100 nucleotides, preferably 17-30 nucleotides.

Also, it is preferred from the viewpoints of the accuracy and sensitivity of PCR detection that the polynucleotides of (c) according to the present invention should each consist of a sequence complementary to the nucleotide sequence of said fusion polynucleotide which extends for not more than 5000 nucleotides from a point of fusion between a polynucleotide encoding FGFR2 protein and a polynucleotide encoding other protein.

The “polynucleotides that are a pair of primers” can be designed by a known method as appropriate based on the nucleotide sequence of the FGFR2 fusion polynucleotide or the like to be targeted. Exemplary known methods include a method using the Primer Express® software (ABI).

Preferred examples of the “polynucleotides that are a pair of primers” are preferably the polynucleotides noted below in (c1) and (c2):

(c1) a combination of a polynucleotide that hybridizes to the portion consisting of the exon 19-coding region of the FGFR2 gene and a region upstream from said coding region toward the 5′ end (this polynucleotide is to be hereinafter also referred to as “5′ FGFR2 primer”), and a polynucleotide that hybridizes to the portion consisting of the exon 3-coding region of the BICC1 gene and a region downstream from said coding region toward the 3′ end (this polynucleotide is to be hereinafter also referred to as “3′ BICC1 primer”); and

(c2) a combination of 5′ FGFR2 primer and a polynucleotide that hybridizes to the portion consisting of the exon 5-coding region of the AHCYL1 gene and a region downstream from said coding region toward the 3′ end (this polynucleotide is to be hereinafter also referred to as “3′ AHCYL1 primer”).

For example, in the process of detection of the FGFR2-BICC1 fusion polynucleotide, primers are each designed for exon 16 or 18 in the kinase region of the FGFR2 gene, or for exon 7/8 or 21 in the BICC1 gene, so that detection can be made of all variants containing the FGFR2 kinase region, or more specifically all fusion genes in which the BICC1 gene is fused with each of the FGFR2 gene isoforms 1-9 which are different from each other in terms of extra-membranous domain structure (e.g., the presence or absence of IgI) (refer to FIG. 2). In the process of detection of the FGFR2-AHCYL1 fusion polynucleotide, primers are each designed for exon 16 or 18 in the kinase region of the FGFR2 gene, or for exon 9 or 20 in the AHCYL1 gene, so that detection can be made of all variants containing the FGFR2 kinase region, or more specifically all fusion genes in which each of the FGFR2 gene isoforms 1-9 which are different from each other in terms of extra-membranous domain structure is fused with each of the AHCYL1 gene isoforms a and b which are different from each other in the length of the N-terminal moiety (refer to FIG. 4).

In the present invention, the method for detecting a translation product of the FGFR2 fusion polynucleotide can be exemplified by immunostaining, Western blotting, ELISA, flow cytometry, immunoprecipitation, and antibody array analysis. These methods use an antibody binding to the FGFR2 fusion polypeptide. Examples of such an antibody include an antibody specific to a polypeptide containing a point of fusion between FGFR2 protein and other protein (hereinafter also referred to as the “fusion point-specific antibody”), an antibody binding to a polypeptide consisting of that region of FGFR2 protein which extends toward the N-terminus with respect to said point of fusion (hereinafter also referred to as the “FGFR2-N terminal antibody”), and an antibody binding to a polypeptide consisting of that region of other protein which extends toward the C-terminus with respect to said point of fusion (hereinafter also referred to as the “other protein-C terminal antibody”). As referred to herein, the “fusion point-specific antibody” means an antibody that specifically binds to a polypeptide containing said point of fusion but does not bind to either wild-type (normal) FGFR2 protein or other wild-type (normal) protein.

The FGFR2 fusion polypeptide can be detected by the fusion point-specific antibody or a combination of the FGFR2-N terminal antibody and the other protein-C terminal antibody.

The “antibody binding to the FGFR2 fusion polypeptide” can be prepared by those skilled in the art through selection of a known method as appropriate. Examples of such a known method include: a method in which the polypeptide comprising the C-terminal moiety of other protein, the FGFR2 fusion polypeptide, the polypeptide comprising the N-terminal moiety of FGFR2 protein, and/or the like are inoculated into immune animals, the immune systems of the animals are activated, and then the serums (polyclonal antibodies) of the animals are collected; as well as monoclonal antibody preparation methods such as hybridoma method, recombinant DNA method, and phage display method. If an antibody having a labeling agent attached thereto is used, a target protein can be directly detected by detecting this label. The labeling agent is not particularly limited as long as it is capable of binding to an antibody and is detectable, and examples include peroxidase, β-D-galactosidase, microperoxidase, horseradish peroxidase (HRP), fluorescein isothiocyanate (FITC), rhodamine isothiocyanate (RITC), alkaline phosphatase, biotin, and radioactive materials. In addition to the direct detection of a target protein using an antibody having a labeling agent attached thereto, the target protein can also be indirectly detected using a secondary antibody having a labeling agent attached thereto, Protein G or A, or the like.

<Method for Determining the Effectiveness of Cancer Treatments with an FGFR2 Inhibitor>

As disclosed below in Examples, gene fusions between the FGFR2 gene and other gene are believed to induce activation of FGFR2 protein and hence contribute to malignant transformation of cancers and other pathological conditions. Thus, it is highly probable that cancer patients with detection of such a fusion are responsive to treatments with an FGFR2 inhibitor.

Therefore, the present invention provides a method for determining the effectiveness of a cancer treatment with an FGFR2 inhibitor, the method comprising the step of detecting the presence or absence of the FGFR2 fusion polynucleotide or the FGFR2 fusion polypeptide in a sample isolated from a patient, wherein in a case where the presence of the polynucleotide or polypeptide is detected, the cancer treatment with the FGFR2 inhibitor is determined to be highly effective in the patient.

For the purpose of the present invention, the “patient” can be not only a human suffering from a cancer but also a human suspected of having a cancer. The “cancer” to which the method of this invention is to be applied is not particularly limited as long as it is a cancer with expression of an FGFR2 fusion gene. The cancer is preferably a biliary tract cancer. Collection of a biological sample from the patient can be performed by a known method depending on the type of the biological sample.

For the purpose of the present invention, the “FGFR2 inhibitor”, the cancer treatment with which is to be evaluated for effectiveness, is not particularly limited as long as it is a substance capable of directly or indirectly suppressing the function of FGFR2 protein. Examples of known FGFR2 inhibitors that can be applied to the present invention include: 3-[2,4-dimethy-5-[[(Z)-2,3-dihydro-2-oxo-1H-indo1-3-ylidene]methyl]-1H-pyrrol-3-yl]propanoic acid (e.g., SU6668 (generic name: orantinib)); N-{5-[2-(3,5-dimethoxyphenyl)ethyl]-1H-pyrazol-3}-yl-4-[(3R,5S)-3,5-dimethylpiperazin-1-yl]benzamide (e.g., AZD4547); 1-[(2R,4S,5S)-4-azido-5-(hydroxymethyl)oxolan-2-yl]-5-methylpyrimidine -2,4-dione (e.g., DS4152 (generic name: tecogalan sodium); FGFR2-IIIb-specific antibody (e.g., AV369b); 3-(2,6-dichloro-3,5-dimethoxyphenyl)-1-[6-[[4-(4-ethylpiperazin-1-yl)phenyl]amino]pyrimidin-4-yl]-1-methylurea (e.g., BGJ398); (S)-(R)-1-((4-((4-fluoro-2-methyl-1H-indol-5-yl)oxy)-5-methylpyrrolo[2,1-f][1,2,4]triazin-6-yl) oxy)propan-2-yl 2-aminopropionate (e.g., BMS-582664 (generic name: brivanib alaninate); and N-[2-[[4-(diethylamino)butyl]amino]-6-(3,5-dimethoxyphenyl)pyrido[2,3-d]pyrimidin-7-yl]-N′-(1,1-dimethylethyl)-urea (e.g., PD173074).

The definition of the term “sample”, the method for extracting a DNA, an RNA or the like from the sample, the procedure for detecting the presence or absence of the FGFR2 fusion polynucleotide or the FGFR2 fusion polypeptide, and other related information are as described above.

If the presence of the FGFR2 fusion polynucleotide or the FGFR2 fusion polypeptide in a sample isolated from a patient is detected according to the inventive method, the patient will be determined to be highly responsive to a cancer treatment with an FGFR2 inhibitor. If the presence of the polynucleotide or polypeptide is not detected, the patient will be determined to be less responsive to a cancer treatment with an FGFR2 inhibitor.

<Agents for Detecting the Presence or Absence of the FGFR2 Fusion Polynucleotide or the FGFR2 Fusion Polypeptide, and Agents for Determining the Effectiveness of Cancer Treatments with an FGFR2 Inhibitor>

As described above, the polynucleotides noted below in (a) to (c), which each have a chain length of at least 15 nucleotides, can be used advantageously for detecting the presence or absence of the FGFR2 fusion polynucleotide. Thus, said polynucleotides can also be used advantageously for determining the effectiveness of cancer treatments with an FGFR2 inhibitor.

(a) A polynucleotide or polynucleotides that are at least one probe selected from the group consisting of a probe that hybridizes to a polynucleotide encoding FGFR2 protein and a probe that hybridizes to a polynucleotide encoding other protein;

(b) a polynucleotide that is a probe that hybridizes to a point of fusion between a polynucleotide encoding FGFR2 protein and a polynucleotide encoding other protein; and

(c) polynucleotides that are a pair of primers designed to sandwich a point of fusion between a polynucleotide encoding FGFR2 protein and a polynucleotide encoding other protein.

Said polynucleotides each have a nucleotide sequence complementary to a particular nucleotide sequence of a target gene. As referred to herein, the term “complementary” may not necessarily refer to perfect complementarity as long as hybridization is achieved. Said polynucleotides have generally at least 80% homology, preferably at least 90% homology, more preferably at least 95% homology, and particularly preferably at least 100% homology with such a particular nucleotide sequence.

The polynucleotides of (a) to (c) may be a DNA or a RNA, or may be such that part or all of the nucleotides are substituted by an artificial nucleic acid such as PNA (polyamide nucleic acid: a peptide nucleic acid), LNA™ (Locked Nucleic Acid; a bridged nucleic acid), ENA® (2′-O,4′-C-Ethylene-bridged Nucleic Acid), GNA (glycerol nucleic acid) or TNA (threose nucleic acid).

As described above, the antibody binding to an FGFR2 fusion polypeptide can be used advantageously for detecting translation products (FGFR2 fusion polypeptides) of the FGFR2 fusion polypeptide.

The agents of the present invention can contain not only the foregoing substance (e.g., polynucleotide, antibody) as an active ingredient but also other pharmacologically acceptable components. Such other components include buffer agents, emulsifying agents, suspending agents, stabilizing agents, antiseptic agents, and physiological saline. As buffer agents, there can be used phosphates, citrates, acetates and the like. As emulsifying agents, there can be used gum arabic, sodium alginate, tragacanth, and the like. As suspending agents, there can be used glyceryl monostearate, aluminum monostearate, methylcellulose, carboxymethyl cellulose, hydroxymethyl cellulose, sodium lauryl sulfate, and the like. As stabilizing agents, there can be used propylene glycol, diethylene sulfite, ascorbic acid, and the like. As antiseptic agents, there can be used sodium azide, benzalkonium chloride, paraoxybenzoic acid, chlorobutanol, and the like.

A specimen containing the inventive polynucleotide or antibody may also be combined with other specimens such as a substrate required for detecting a label attached to the polynucleotide or the antibody, a positive control (e.g., FGFR2 fusion polynucleotide, FGFR2 fusion polypeptide, or cells bearing the same), a negative control, a counterstaining reagent for use for in situ hybridization or the like (e.g., DAPI), a molecule required for antibody detection (e.g., secondary antibody, Protein G, Protein A), and a buffer solution for use in sample dilution or washing, so that a kit for use in the method of the present invention can be provided. The inventive kit can contain instructions for use thereof. Therefore, the present invention also provides the foregoing kit for use in the inventive method.

<Method for Treatment of Cancer, and Therapeutic Agents for Cancer>

As described above, if the presence of the FGFR2 fusion polynucleotide or the FGFR2 fusion polypeptide is detected in a patient by the method of the present invention, the patient is considered to be highly responsive to a cancer treatment with an FGFR2 inhibitor. Thus, efficient cancer treatment is possible by administering an FGFR2 inhibitor selectively to those cancer patients who carry the FGFR2 fusion gene. Therefore, this invention provides a method for treatment of cancer, comprising the step of administering an FGFR2 inhibitor to a patient in whom a cancer treatment with the FGFR2 inhibitor has been determined to be highly effective according to the foregoing determination method of this invention.

Further, the present invention provides a therapeutic agent for cancer, comprising an FGFR2 inhibitor as an active ingredient, the agent which is to be administered to a patient in whom a cancer treatment with the FGFR2 inhibitor has been determined to be highly effective according to the foregoing determination method of this invention.

As described above, the “FGFR2 inhibitor” is not particularly limited as long as it is a substance capable of directly or indirectly suppressing the function of FGFR2 protein. Examples of known FGFR2 inhibitors that can be applied to the present invention are as given above.

The dosage form for administering an FGFR2 inhibitor to a patient is selected as appropriate depending on various factors including the type of the inhibitor and the type of cancer, and examples of the dosage form that can be adopted include oral, intravenous, intraperitoneal, transdermal, intramuscular, intratracheal (aerosol), rectal, intravaginal and other administrations.

### EXAMPLES

On the pages that follow, the present invention will be more specifically described on the basis of Examples, but this invention is not limited to the examples given below.

<Test Samples>

Eight surgically resected and frozen specimens of KRAS/BRAF mutation-negative biliary tract cancer (tumor tissues) were used as an object to be tested. As a negative control, normal tissues (liver tissues containing biliary epithelium) were also treated and analyzed by the methods described below as in the case of the tumor tissues.

<RNA Extraction>

The tumor tissues cryopreserved in liquid nitrogen were pulverized with cryoPREP (product name: CP02; Covaris). Then, total RNA was extracted from the pulverized tumor tissues using a total RNA purification kit (product name: RNAeasy; QIAGEN).

<RNA Sequencing>

Library synthesis was performed with an mRNAseq sample preparation kit (Illumina) using 2 μg of the total RNA prepared hereinabove. More specifically, 2 μg of the total RNA was fragmented by treatment at 94° C. for 5 minutes and subjected to cDNA synthesis. Next, a sequencing adapter was ligated to each end of the resulting cDNA, which was then subjected to electrophoresis on agarose gel and purified. PCR was performed using the purified cDNA as a template to construct a 300 by cDNA library. The sequences of 50 by from both ends of the constructed cDNA library were sequenced using a high-throughput sequencer (GA2X; Illumina).

<Analysis of Sequence Information>

The obtained sequence information, from which overlapping clones generated by PCR were excluded, was mapped to known databases (Refseq and Ensemble) using the Bowtie software to extract clones whose end sequences are derived from different genes (refer to Kohno T., et al., Nature Med., 2012, vol. 18, p. 375-377).

<Verification by RT-PCR and Sanger method>

The same total RNA as used in RNA sequencing was subjected to cDNA synthesis anew using a reverse transcriptase (SuperScript III First-Strand Synthesis System; Invitrogen). PCR primers were synthesized based on the resulting sequences. PCR was performed using ExTaq HS (Takara), and then the PCR products were verified by electrophoresis. Further, the PCR products were extracted from the agarose gel and sequenced by the Sanger method using the same primers, the BigDye Terminator v3.1 Cycle Sequencing Kit (Applied Biosystems) and the ABI 3730 Sequencer. The obtained results were used to identify points of fusion of fusion genes and reading frames. The conditions for reverse transcription reaction and PCR are as follows.

<Reverse Transcription Reaction>

First, 5 μg (8 μL) of the total RNA was mixed with 1 μL of Random Hexamer Primer (50 ng/μL) and 1μL of 10 mM dNTP Mix, and the mixture was reacted at 65° C. for 5 minutes and quenched on ice. Next, 2 μL of 10×RT buffer, 4 μL of 25 mM MgCl₂, 2 μL of 0.1M DTT, 1 μL of RNaseOUT (40 U/μL), and 1 μL of SuperScript III RT (200 U/μL) were added in this order, and reverse transcription reaction was effected under the following conditions: 25° C. for 10 minutes, 50° C. for 50 minutes, 85° C. for 5 minutes, and 4° C. for 5 minutes. To the resulting reverse transcripts, 1 μL of RNaseH was added to digest the total RNA at 37° C. over 20 minutes. The completed reverse transcription reactions were stored at −20° C. until use.

<PCR>

First, 2 μL of the hereinabove prepared 1st strand cDNA, 1 μL of 10×ExTaq buffer, 1.2 μL of 2.5 mM dNTPs, 4.7 μL of H₂O, 0.1 μL of ExTaq HS, and 1 μL of 2 μM CF/CR primer pair were mixed, and the mixture was subjected to PCR under the following conditions: the reaction started with 95° C. for 3 minutes, followed by 35 cycles consisting of 94° C. for 30 seconds, 58° C. for 30 seconds, and 72° C. for 30 seconds, and ended with 72° C. for 5 minutes.

### Example 1

[Identification of Novel Kinase Fusion Genes by RNA Sequencing]

From each of 8 KRAS/BRAF-negative BTC clinical specimens, there were obtained at least 5.2×10⁷ paired nucleotide sequences, with overlapping clones generated by PCR being excluded. As the result of comparison of these sequences to existing gene databases, the following two fusion gene candidates as shown in FIGS. 2 and 4 were detected each in one specimen: a fusion gene between the FGFR2 gene and the BICC1 gene (hereinafter also referred to the “FGFR2-BICC1 fusion gene”), and a fusion gene between the FGFR2 gene and the AHCYL1 gene (hereinafter also referred to as the “FGFR2-AHCYL1 fusion gene”).

[Verification by RT-PCR and Sanger Sequencing]

Next, the same specimens were verified for fusion genes by RT-PCR and Sanger sequencing. As a result, RT-PCR revealed that specimen-specific amplification is observed for both of the two fusion genes, or in other words that these fusion genes are expressed only in BTC tissues and not in normal tissues (liver tissues containing biliary epithelium).

Further, sequencing of the obtained PCR products revealed that in both of these fusion genes, two genes are fused together without inconsistency in their reading frames.

More specifically, it was found that in the fusion gene between the FGFR2 gene and the BICC1 gene, as shown in FIG. 2 and SEQ ID NO: 7, exon 19 of the FGFR2 gene (isoform 1) (i.e., the polynucleotide consisting of the nucleotide sequence of positions 2843 to 2948 in SEQ ID NO: 1 or 7) was directly bound and fused to exon 3 of the BICC1 gene (i.e., the polynucleotide consisting of the nucleotide sequence of positions 238 to 307 in SEQ ID NO: 3) without inconsistency in the reading frames of these genes.

It was also found that in the fusion gene between the FGFR2 gene and the AHCYL1 gene, as shown in FIG. 4 and SEQ ID NO: 9, exon 19 of the FGFR2 gene (isoform 1) (i.e., the polynucleotide consisting of the nucleotide sequence of positions 2843 to 2948 in SEQ ID NO: 1 or 7) was directly bound and fused to exon 5 of the AHCYL1 gene (isoform a) (i.e., the polynucleotide consisting of the nucleotide sequence of positions 410 to 521 in SEQ ID NO: 5) without inconsistency in the reading frames of these genes.

Accordingly, the results presented hereinabove show that the inversion in chromosome 10 and the reciprocal translocation between chromosome 1 and chromosome 10, which are specific to biliary tract and other cancer cells, result in a fusion between the FGFR2 gene and the BICC1 gene, and a fusion between the FGFR2 gene and the AHCYL1 gene, respectively.

### Example 2

[Analysis of the Appearance Frequencies of the FGFR2 Fusion Genes]

The hereinabove-identified cancer cell-specific fusion genes, FGFR2-AHCYL1 and FR2-BICC1 fusion genes, were each analyzed for their appearance frequencies. More specifically, 102 cancer tissues derived from biliary tract cancer (BTC) patients were subjected to RT-PCR analysis of the appearance frequencies of the respective fusion genes.

As a result, among the 102 BTC specimens, the FGFR2/AHCYL1 fusion was observed in 7 specimens (appearance frequency: about 6.9%), and the FGFR2/BICC1 fusion was observed in 2 specimens (appearance frequency: about 2.0%).

### Example 3

[Analyses of the Function of the FGFR2 Fusion Polypeptides, and of the Effectiveness of FGFR Inhibitors Against Cancer Cells Expressing Said Polypeptides]

It is considered that the above-mentioned gene fusions induce activation of FGFR2 protein, and that this activation induces activation of a downstream signal, thereby causing canceration of cells. Therefore, it is conceivable that FGFR2 inhibitors may be therapeutically effective in patients with such activations. In order to verify these points, analysis of the FGFR2 fusion genes was made by following appropriate conventional methods, as described below.

First, a cDNA encoding a FLAG epitope tag was joined to the 5′ end of each of the cDNAs of the FGFR2 fusion genes obtained from BCT patients' cancer tissues (FGFR2-AHCYL1 fusion gene cDNA or FGFR2-BICC1 fusion gene cDNA), by aligning their translated reading frames with each other. The resulting products were each cloned into the pMXs retroviral vector.

Also, site-directed mutagenesis was performed on the vectors to construct vectors encoding a kinase activity mutant containing a substitution of 2 amino acids in an FGFR2 kinase region (KD-mutated FGFR2 fusion polypeptides: Y568F/Y569F).

Next, normal murine fibroblast line NIH-3T3 cells were infected with each of the thus-prepared retroviral vectors to obtain cell lines stably expressing the wild-type or the mutated FGFR2 fusion polypeptide.

The same procedures were performed for a tyrosine kinase fusion gene detected in lung cancer (fusion gene between the EZR gene and the ROS 1 gene; hereinafter also referred to as the “EZR-ROS1 fusion gene”)—a retroviral vector encoding this gene was constructed, and NIH-3T3 cells were infected with this vector to prepare a cell line stably expressing the EZR-ROS1 fusion polypeptide. This cell line was subjected to the tests described below as a control group.

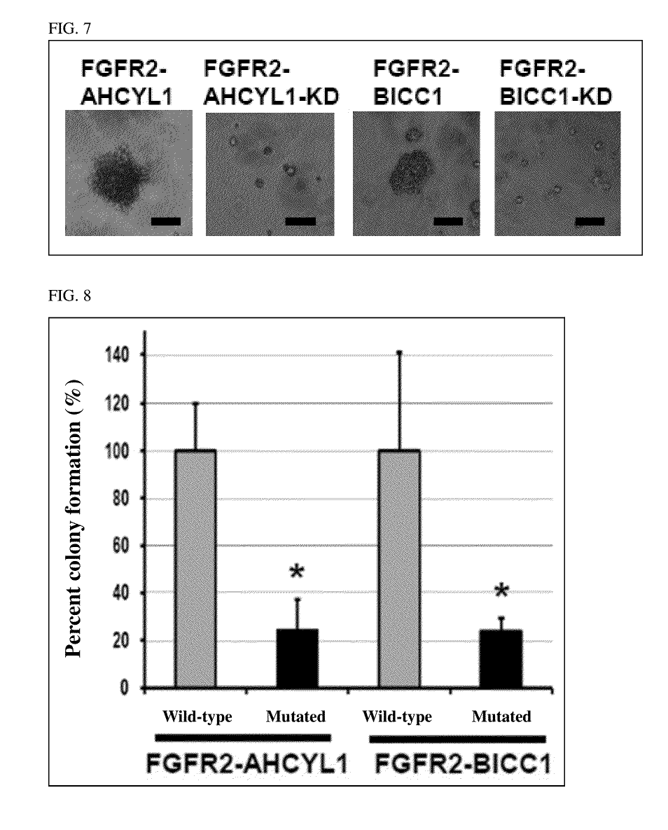

The cell lines stably expressing the wild-type or the mutated FGFR2 fusion polypeptide were each seeded in a soft agar medium (at an agar concentration in medium of 4 mg/mL; the same applies hereunder) and evaluated for their anchorage-independent colony-forming ability to thereby investigate the transforming ability of the FGFR2 fusion polypeptides. The results are shown in FIGS. 7 and 8.

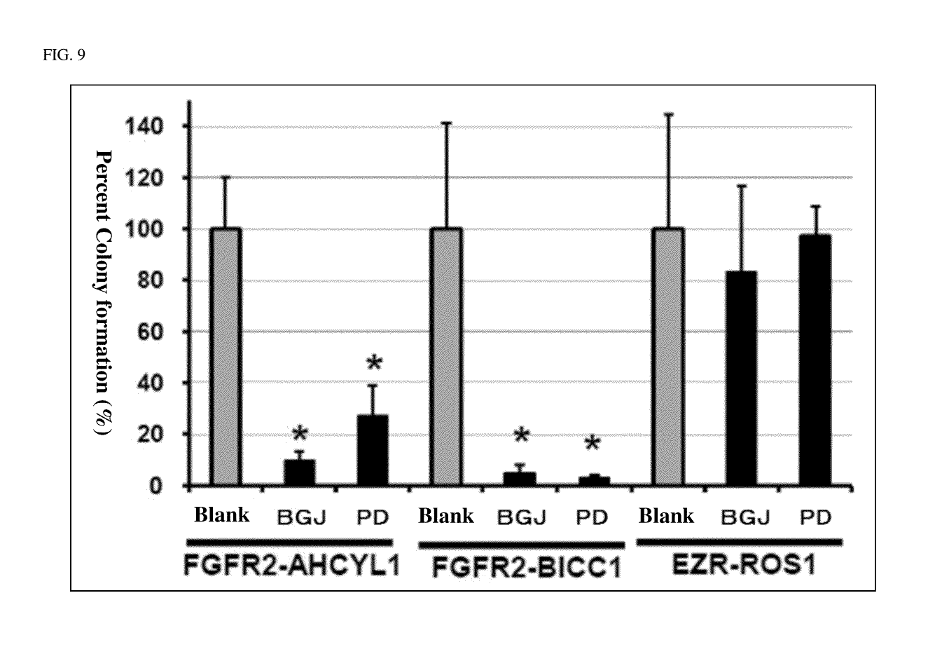

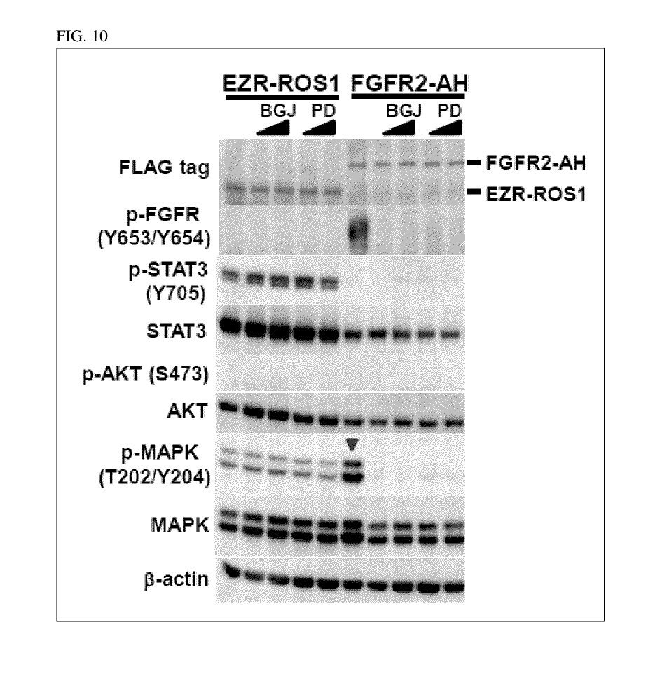

Further, the cell lines stably expressing the wild-type FGFR2 fusion polypeptides and the cell line stably expressing the EZR-ROS 1 fusion polypeptide were each seeded in a soft agar medium supplemented with a low-molecular-weight FGFR kinase inhibitor (BGJ398 or PD173074) and evaluated for their anchorage-independent colony-forming ability to thereby investigate the effect of FGFR kinase inhibitors against transformation induced by the FGFR2 fusion polypeptides. The results are shown in FIG. 9.

In addition, the cell line stably expressing the wild-type FGFR2-AHCYL1 fusion polypeptide and the cell line stably expressing the EZR-ROS 1 fusion polypeptide were each cultured in a liquid medium, subjected to serum starvation, and then cultured again with the medium being replaced with the one supplemented with an FGFR kinase inhibitor (BGJ398 or PD173074). Proteins from each of the thus-obtained cell lines were extracted and subjected to Western blotting analysis using antibodies against phosphorylated or unphosphorylated proteins to thereby analyze the downstream signals of the FGFR2 fusion polypeptide. The results are shown in FIG. 10.

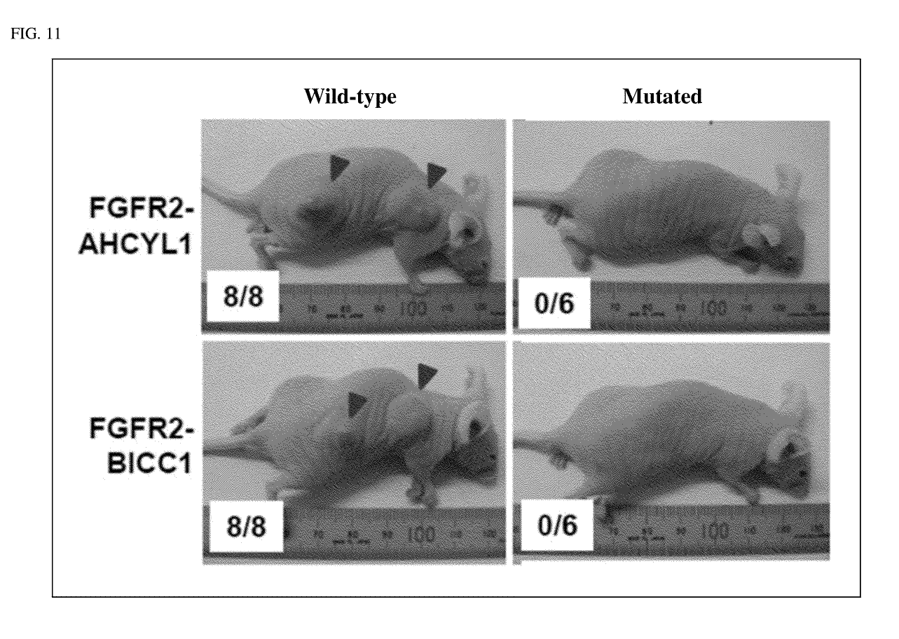

Furthermore, the cell lines stably expressing the wild-type or the mutated FGFR2 fusion polypeptide were each subcutaneously transplanted into immunodeficient mice (BALB/c-nu/nu) at a dose of 1×10⁶ cells per spot to thereby investigate the in vivo tumorigenic ability of the cells expressing these FGFR2 fusion polypeptides. The results are shown in FIG. 11.

As is evident from the results shown in FIGS. 7 and 8, NIH-3T3 cells showed anchorage-independent colony formation due to the expression of the FGFR2 fusion polypeptides. On the other hand, it was found that the anchorage-independent colony-forming ability of the FGFR2 fusion polypeptides is significantly suppressed by inactivating the kinase activity of FGFR2 protein.

It was also found as shown in FIG. 9 that the anchorage-independent colony-forming ability of the FGFR2 fusion polypeptides is significantly suppressed by using an FGFR kinase inhibitor.

As is evident from the results given in FIG. 10, NIH-3T3 cells showed strong phosphorylation of the FGFR kinase domain due to the expression of the FGFR2 fusion polypeptide (refer to the 5th lane from the right in the “p-FGFR (Y653/Y654)” row of FIG. 10). It was also found that this phosphorylation is significantly suppressed by treatment with an FGFR kinase inhibitor (refer to the 4th to 1st lanes from the right in the “p-FGFR (Y653/Y654)” row of FIG. 10).

As shown in FIG. 10, the phosphorylation of MAPK was also strongly induced by the FGFR2 fusion polypeptide. However, it was found that this phosphorylation is also significantly suppressed by treatment with an FGFR kinase inhibitor (refer to the 5th lane v.s. the 4th to 1st lanes from the right in the “p-MAPK (T202/Y204)” row of FIG. 10). Additionally, the phosphorylations of AKT and STATS were not increased by the expression of the FGFR2 fusion polypeptide.

As shown in FIG. 11, tumorigenesis was observed within 14 days after NIH-3T3 cells expressing the wild-type FGFR2 fusion polypeptide were subcutaneously transplanted into immunodeficient mice. On the other hand, NIH-3T3 cells expressing the mutated FGFR2 fusion polypeptide were also subcutaneously transplanted into immunodeficient mice, but no tumorigenesis was observed at all for 30 days after the transplantation.

Accordingly, these findings demonstrated that the FGFR2 fusion genes serve as an oncogene with transforming ability, and that their transforming ability requires the activation of the FGFR2 kinase activity.

It is also found that the transforming ability of said FGFR2 fusion polypeptides is suppressed using an FGFR kinase inhibitor by inhibiting the activations of the FGFR2 kinase in said fusion polypeptides and its downstream signal, MAPK.

### INDUSTRIAL APPLICABILITY

As described above, the present invention enables detection of polynucleotides encoding fusion polypeptides between FGFR2 protein and other protein, and expression products thereof, and also this detection makes it possible to predict the effectiveness of cancer treatments with an FGFR2 inhibitor. As described above, an in-frame fusion between the FGFR2 gene and other gene was found in multiple cases, and this fusion induces the activation of FGFR2, and in turn causes canceration of cells. Further, as demonstrated above, said FGFR2 activation and canceration can be significantly suppressed by using an FGFR2 inhibitor. Therefore, since such fusions between the FGFR2 gene and other gene can be targeted by FGFR2 inhibitors, the present invention is very useful in improving the efficiency of cancer treatments.

1. A polynucleotide encoding a polypeptide in which FGFR2 protein and a second protein are fused together, wherein the polypeptide is expressed in a cancer cell.

2. The polynucleotide according to claim 1, wherein said second protein is BICC1 protein or AHCYL1 protein.

3. The polynucleotide according to claim 1, wherein the cancer cell is a biliary tract cancer cell.

4. A polypeptide encoded by the polynucleotide according to claim 1.

5. A method for detecting the presence or absence in a sample of the polynucleotide according to claim 1, the method comprising the steps of:
(a) contacting the sample with an agent intended for specifically detecting the presence or absence of the polynucleotide in the sample; and
(b) detecting the presence or absence of the polynucleotide.

6. An agent for detecting the presence or absence in a sample of a polynucleotide encoding a polypeptide in which FGFR2 protein and a second protein are fused together for use in the method according to claim 5, the agent comprising a polynucleotide or polynucleotides as set forth below in any one of (a) to (c), the polynucleotide or polynucleotides having a chain length of at least 15 nucleotides, or an antibody as set forth below in (d):
(a) a polynucleotide or polynucleotides that are at least one probe selected from the group consisting of a probe that hybridizes to a polynucleotide encoding FGFR2; protein and a probe that hybridizes to a polynucleotide encoding the second protein;
(b) a polynucleotide that is a probe that hybridizes to a point of fusion between a polynucleotide encoding FGFR2 protein and a polynucleotide encoding the second protein;
(c) polynucleotides that are a pair of primers designed to sandwich a point of fusion between a polynucleotide encoding FGFR2 protein and a polynucleotide encoding the second protein; and
(d) an antibody that binds to a polypeptide in which FGFR2 protein and the Second protein are fused together.

7. A method for determining the effectiveness of a cancer treatment with an FGFR2 inhibitor, the method comprising the step of detecting the presence or absence in a sample isolated from a patient of the polynucleotide according claim 1, wherein in a case where the presence of the polynucleotide is detected, the cancer treatment with the FGFR2 inhibitor is determined to be highly effective in the patient.

8. An agent for determining the effectiveness of a cancer treatment with an FGFR2 inhibitor by the method according to claim 7, the agent comprising a polynucleotide or polynucleotides as set forth below in any one of (a) to (c), the polynucleotide or polynucleotides having a chain length of at least 15 nucleotides, or an antibody as set forth below in (d):
(a) a polynucleotide or polynucleotides that are at least one probe selected from the group consisting of a probe that hybridizes to a polynucleotide encoding FGFR2 protein and a probe that hybridizes to a polynucleotide encoding the second protein;
(b) a polynucleotide that is a probe that hybridizes to a point of fusion between a polynucleotide encoding FGFR2 protein and a polynucleotide encoding the second protein;
(c) polynucleotides that are a pair of primers designed to sandwich a point of fusion between a polynucleotide encoding FGFR2 protein and a polynucleotide encoding the second protein; and
(d) an antibody that binds to a polypeptide in which FGFR2 protein and the second protein are fused together.

9. A method for treatment of cancer, comprising the step of administering an FGFR2 inhibitor to a patient in whom a cancer treatment with the FGFR2 inhibitor has been determined to be highly effective by the method according to claim 7.

10. A therapeutic agent for cancer, comprising an FGFR2 inhibitor as an active ingredient, the agent which is to be administered to a patient in whom a cancer treatment with the FGFR2 inhibitor has been determined to be highly effective by the method according to claim 7.

11. A method for detecting the presence or absence in a sample of the polypeptide according to claim 4, the method comprising the steps of:
(a) contacting the sample with an agent intended for specifically detecting the presence or absence of the polypeptide in the sample; and
(b) detecting the presence or absence of the polypeptide.

12. The method of claim 5 wherein the second protein is either BICC1 protein or AHCYL1 protein.

13. An agent for detecting the presence or absence in a sample of a polypeptide in which FGFR2 protein and a second protein are fused together for use in the method according to claim 11, the agent comprising a polynucleotide or polynucleotides as set forth below in any one of (a) to (c), the polynucleotide or polynucleotides having a chain length of at least 15 nucleotides, or an antibody as set forth below in (d):
(a) a polynucleotide or polynucleotides that are at least one probe selected from the group consisting of a probe that hybridizes to a polynucleotide encoding FGFR2; protein and a probe that hybridizes to a polynucleotide encoding the second protein;
(b) a polynucleotide that is a probe that hybridizes to a point of fusion between a polynucleotide encoding FGFR2 protein and a polynucleotide encoding the second protein;
(c) polynucleotides that are a pair of primers designed to sandwich a point of fusion between a polynucleotide encoding FGFR2 protein and a polynucleotide encoding the second protein; and
(d) an antibody that binds to a polypeptide in which FGFR2 protein and the second protein are fused together.

14. The method of claim 11 wherein the second protein is either BICC1 protein or AHCYL1 protein.

15. A method for determining the effectiveness of a cancer treatment with an FGFR2 inhibitor, the method comprising the step of detecting the presence or absence in a sample isolated from a patient of the polypeptide according to claim 4, wherein in a case where the presence of the polypeptide is detected, the cancer treatment with the FGFR2 inhibitor is determined to be highly effective in the patient.

16. An agent for determining the effectiveness of a cancer treatment with an FGFR2 inhibitor by the method according to claim 15, the agent comprising a polynucleotide or polynucleotides as set forth below in any one of (a) to (c), the polynucleotide or polynucleotides having a chain length of at least 15 nucleotides, or an antibody as set forth below in (d):
(a) a polynucleotide or polynucleotides that are at least one probe selected from the group consisting of a probe that hybridizes to a polynucleotide encoding FGFR2 protein and a probe that hybridizes to a polynucleotide encoding the second protein;
(b) a polynucleotide that is a probe that hybridizes to a point of fusion between a polynucleotide encoding FGFR2 protein and a polynucleotide encoding the second protein;
(c) polynucleotides that are a pair of primers designed to sandwich a point of fusion between a polynucleotide encoding FGFR2 protein and a polynucleotide encoding the second protein; and
(d) an antibody that binds to a polypeptide in which FGFR2 protein and the second protein are fused together.

17. A method for treatment of cancer, comprising the step of administering an FGFR2 inhibitor to a patient in whom a cancer treatment with the FGFR2 inhibitor has been determined to be highly effective by the method according to claim 15.

18. A therapeutic agent for cancer, comprising an FGFR2 inhibitor as an active ingredient, the agent which is to be administered to a patient in whom a cancer treatment with the FGFR2 inhibitor has been determined to be highly effective by the method according to claim 15.
